(19) United States
(12) Patent Application Publication
(10) Pub. No.: US 2025/0215028 A1
(43) Pub. Date: Jul. 3, 2025

(54) SYNTHETIC GLYCEROPHOSPHOLIPIDS COMPRISING AT LEAST ONE REACTIVE FUNCTION, PROCESS FOR PREPARING SAME AND USES THEREOF IN DIFFERENT APPLICATIONS

(71) Applicants: UNIVERSITE DE ROUEN-NORMANDIE, Mont-Saint-Aignan (FR); CENTRE NATIONAL DE LA RECHERCHE SCIENTIFIQUE, Paris (FR); INSA ROUEN NORMANDIE, Saint Etienne du Rouvray Cedex (FR); INSTITUT NATIONAL DE LA SANTE ET DE LA RECHERCHE MEDICALE, Paris (FR)

(72) Inventors: Sébastien BALIEU, Notre-Dame de Bondeville (FR); Pierre-Yves RENARD, Paris (FR); Maité MONTERO-HADJADJE, Rouen (FR); Nicolas VITALE, Strasbourg (FR); Aurélien COCQ, Bihorel (FR); Antoine SCHLICHTER, Rouen (FR); Rémy FERLET, Villenave d'ornon (FR); Alexandre HAEFELE, Mont-Saint-Aignan (FR); Lina RIACHY, Rouen (FR); Alexander WOLF, Strasbourg (FR)

(21) Appl. No.: 18/850,339

(22) PCT Filed: Mar. 23, 2023
(86) PCT No.: PCT/EP2023/057500

§ 371 Date: Sep. 24, 2024

(30) Foreign Application Priority Data
Mar. 24, 2022 (FR) FR2202640

(51) Int. Cl. C07F 9/6558; C07F 9/09; C07F 9/117; G01N 33/532

(52) U.S. Cl. CPC C07F 9/65586; C07F 9/091; C07F 9/117; G01N 33/532

(57) ABSTRACT

The present invention applies to the field of glycerophospholipids (GPL) that may be used for biological studies. In particular, the invention relates to new GPL compounds comprising at least one reactive function which does not interfere with biological processes, to the process for preparing them, and to their uses in various applications.

The present invention applies to the field of glycerophospholipids (GPL) that may be used for biological studies. In particular, the invention relates to new GPL compounds comprising at least one reactive function which does not interfere with biological processes, to the process for preparing them, and to their uses in various applications.

Glycerophospholipids (GPL) represent the most abundant class of cellular lipids. They are also the main constituents of membrane bilayers and are involved in numerous cellular processes such as mobility, energy production and intracellular trafficking. In particular, they are directly involved in numerous intracellular signaling pathways. They are amphiphilic lipids, meaning that they have a hydrophilic part (polar head) and a hydrophobic part (apolar tail). GPLs are built around a glycerol molecule, where two hydroxyl groups are esterified with different fatty acids and the third hydroxyl group is replaced with phosphoric acid.

A natural mammalian GPL may be represented by the structural formula below:

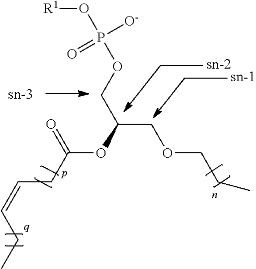

in which there is a glycerol backbone onto which are grafted:
in the “sn-3” position of the glycerol backbone, a polar phosphate head, which may be functionalized (R¹≠H) or not (R¹=H),
in the “sn-2” position of the glycerol backbone, a mono- or polyunsaturated unsaturated fatty acid chain (p+q+3 carbon atoms) (not represented), of variable length, and
in the “sn-1” position of the glycerol backbone, generally a saturated fatty acid chain (n+1 carbon atoms) of variable length.

The polar phosphate head in the sn-3 position gives GPLs their generic name and some of their biological properties. There are some twenty sub-classes of GPLs, depending on the type of group R¹ present on the polar head: the most abundant GPL is phosphatidylcholine (PC), which bears a choline head. Other GPLs are also naturally present, such as phosphatidylethanolamine (PE), phosphatidylserine (PS), phosphatidylinositol (PI), phosphatidic acid (PA) and phosphatidylglycerol (PG).

A cell may contain several thousand different phospholipids. As their metabolism is rapid and often complex, it proves to be extremely difficult to know the precise location and role of any given phospholipid at any given instant.

Research teams have consequently turned their attention to the synthesis of probes based on glycerophospholipid structures, for example containing fluorescent groups. Some of these probes are moreover sold and are represented in the scheme below. In particular, the fluorophoric group (represented in a box) is added either by replacing part of a fatty acid chain (18:0-6:0 NBD PA and TOPFLUOR PI(4,5)P₂), or on the phosphate polar head ((18:1)₂ DANSYL PS).

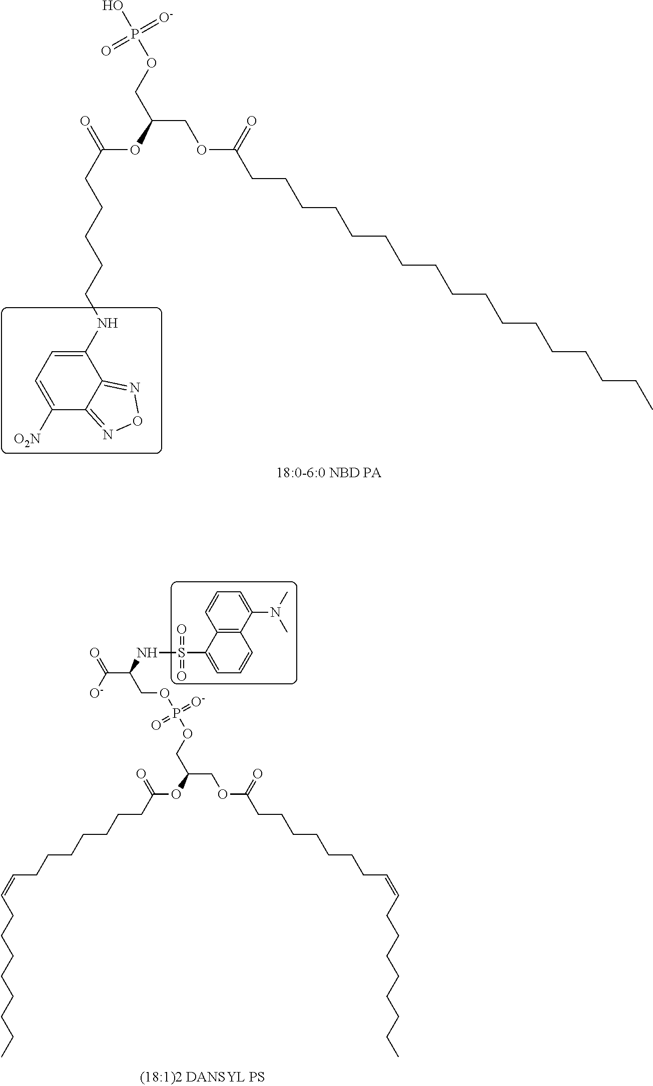

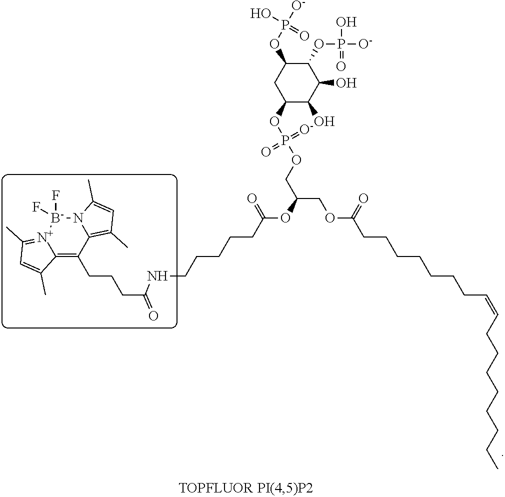

Other probes are also described in patent application NZ577912 A. Said patent application relates to water-soluble fluorescent cell markers corresponding to the structure F-S1-S2-L, in which F is a fluorophore, S1-S2 is a spacer arm linking F to L, L is a lipid chosen from diacyl and dialkyl glycerophospholipids, and the F-S1-S2-L structure includes the following substructure —C(═R)—NH—(CH₂)ₘ—NH—C(═O)—(CH₂)ₙ—C(═O)— in which m and n independently range from 3 to 6 and R is O or S. In the examples, the fluorophore is positioned at the phosphate polar head via the S1-S2 linker spacer arm. One of the examples has the following structure:

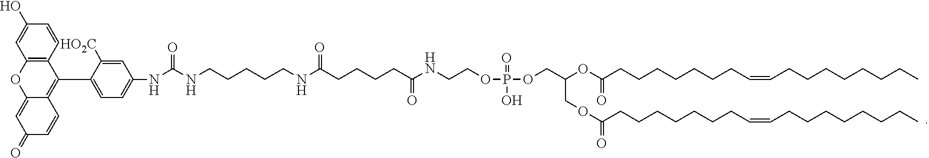 [SMILES: CCCCCCCC/C=C\CCCCCCCC(=O)OCC(COP(=O)(O)OCCNC(=O)CCCCC(=O)NCCCCCNC(=O)Nc1ccc(-c2c3ccc(=O)cc-3oc3cc(O)ccc23)c(C(=O)O)c1)OC(=O)CCCCCCC/C=C\CCCCCCCC]

However, the abovementioned probes have the drawback of being too far removed in terms of structure from the molecules whose subcellular distribution they are intended to illustrate. Specifically, the polar heads of glycerophospholipids ensure the specificity of interaction with certain proteins, and the nature of the fatty acid chains also plays an important role in the functions of GPLs, and in interactions with proteins. Consequently, any modifications in these areas (polar head or fatty acids) clearly jeopardize the recognition of GPLs by their mobilizing proteins, potentially modifying their biological functions, and thus biasing or rendering impossible interpretations of experiments performed with these probes. Moreover, these probes are synthesized with a particular fluorophore, making it necessary to synthesize a new probe with a different fluorophore in order to vary the probe's photophysical properties if required.

Consequently, there is a need for new glycerophospholipid probes with a structure closer to that of natural GPLs, which are easy to prepare, and which can be used in a variety of applications and in particular as molecular tools in the field of biology.

Thus, the aim of the present invention is to overcome the drawbacks of the abovementioned prior art and to provide synthetic glycerophospholipids in which the main features of natural glycerophospholipids are conserved while at the same time ensuring their use as molecular tools in the field of biology. Another aim of the present invention is to provide a process for preparing such synthetic glycerophospholipids which is simple, convergent, economical, and affords access to glycerophospholipids of varied structures.

The aim of the invention is achieved via the compounds and process that will be described hereinbelow.

A first subject of the present invention is thus a synthetic glycerophospholipid corresponding to formula (I) below:

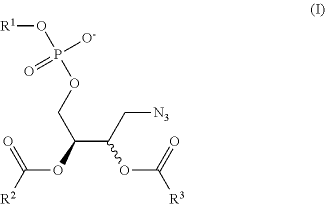

in which:
the group OR¹ is chosen from a hydroxyl group, a choline residue, an ethanolamine residue, a glycerol residue, a serine residue, an inositol residue, an inositol monophosphate residue, an inositol bisphosphate residue, an inositol trisphosphate residue, a glycerophosphate residue, a glucose residue, an inositolglycan residue, and a residue of any hydroxylated compound that is capable of forming a phosphodiester group in the sn-3 position belonging to a subclass of natural GPLs,
R² represents a mono- or polyunsaturated, unsaturated aliphatic chain comprising at least 14 carbon atoms, and
R³ represents a saturated aliphatic chain comprising at least 10 carbon atoms.

The GPLs of formula (I) of the invention respect the nature of the lipid chains (i.e. a saturated fatty acid chain in position sn-1 and an unsaturated fatty acid chain in position sn-2) and of the phosphate polar head in position sn-3, which affords access to molecular tools with improved reliability. Moreover, they bear a small-sized discrete azide function in the “sn-0” position, which does not significantly modify their properties and may serve as a binding point for a marker or any biological probe, notably by click chemistry. This discrete azide function can thus afford suitable molecular tools for fine exploration of the localization and various in vitro, in vivo or in cellulo roles of GPLs. Firstly, it does not interfere with biological processes, which should allow the biological properties of GPLs to be conserved irrespective of the lipid chains, and consequently the role of the different chains present in GPLs to be precisely understood. Secondly, this discrete azide function may then be used once the GPL of formula (I) is introduced in vitro, in vivo or in cellulo to graft various chemical tools (fluorophore, photoactivatable crosslinking agent, radiolabel, etc.) enabling biological studies without having to recommence a specific synthesis of the glycerophospholipid backbone.

For the purposes of the present invention, the term “residue” of a hydroxylated compound means this same hydroxylated compound in which the hydrogen atom of the hydroxyl function of said hydroxylated compound has been removed to form the group OR¹ of the phosphodiester. By way of example, a choline residue is the group OR¹=—O—(CH₂)₂—N+(CH₃)₃, choline corresponding to the hydroxylated compound H—O—(CH₂)₂—N+(CH₃)₃.

As explained in the introductory section, there are some twenty sub-classes of natural GPLs in mammals, depending on the type of group R¹ present on the polar head.

For the purposes of the present invention, the compound of formula (I) comprises two chiral centers, the first in position sn-2 whose configuration is fixed in such a way as to correspond to that of natural GPLs, and the second in position sn-1 which has two possible configurations. Said compound of formula (I) may thus be chosen from a compound of formula (I)-C1, a compound of formula (I)-C2, and a mixture of the compounds of formula (I)-C1 and of formula (I)-C2, said formulae (I)-C1 and (I)-C2 being as defined below:

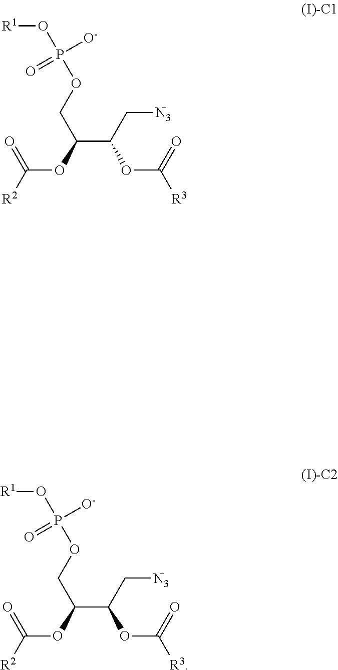

Preferably, compound (I) is a compound (I)-C1.

### Definition of R¹

The group OR¹ is preferably chosen from a hydroxyl group, a choline residue, an ethanolamine residue, a glycerol residue, a serine residue and an inositol residue.

### Definition of R²

R² represents a mono- or polyunsaturated unsaturated aliphatic chain comprising at least 14 carbon atoms.

It preferably comprises from 16 to 24 carbon atoms and particularly preferably from 16 to 20 carbon atoms.

The unsaturated aliphatic chain may comprise from 1 to 6 unsaturations, preferably from 1 to 3 unsaturations.

Each of the unsaturations may be of Z or E configuration.

The unsaturated aliphatic chain is preferably an unsubstituted unsaturated aliphatic chain.

The unsaturated aliphatic chain is preferably a linear (i.e. unbranched) unsaturated aliphatic chain.

### Definition of R³

R³ represents a saturated aliphatic chain comprising at least 10 carbon atoms.

It preferably comprises from 14 to 30 carbon atoms and particularly preferably from 14 to 24 carbon atoms.

The saturated aliphatic chain is preferably an unsubstituted saturated aliphatic chain.

The saturated aliphatic chain is preferably a linear (i.e. unbranched) saturated aliphatic chain.

According to a preferred embodiment of the invention, the glycerophospholipid (GPL) is chosen from the compounds of formulae (Ia) to (Ie) shown in the following table:

| Name | Structural formula |
|---|---|
| Compound Ia | 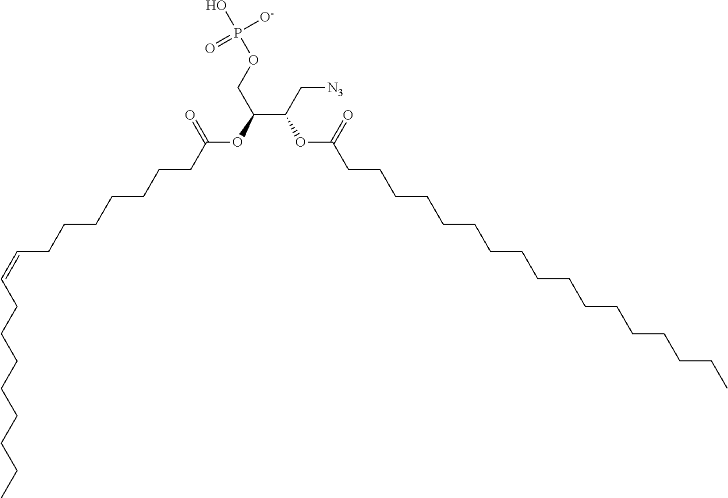 [SMILES: CCCCCCCC/C=C\CCCCCCCC(=O)O[C@@H](COP(=O)([O-])O)[C@H](CN=[N+]=[N-])OC(=O)CCCCCCCCCCCCCCCCC] |
| Compound Ib | 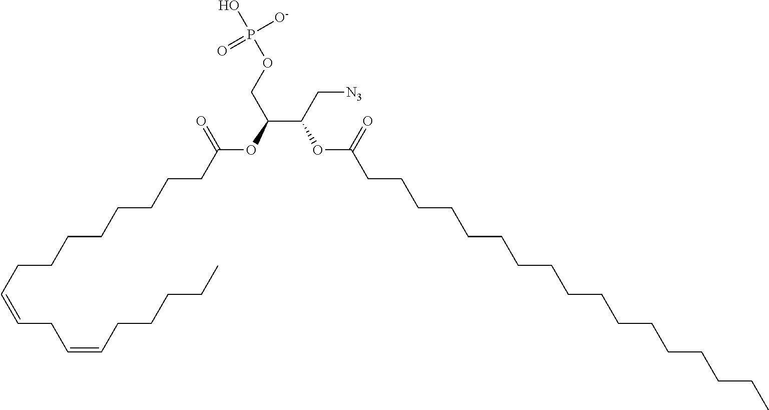 [SMILES: CCCCC/C=C\C/C=C\CCCCCCCCC(=O)O[C@@H](COP(=O)([O-])O)[C@H](CN=[N+]=[N-])OC(=O)CCCCCCCCCCCCCCCCC] |
| Compound Ic | 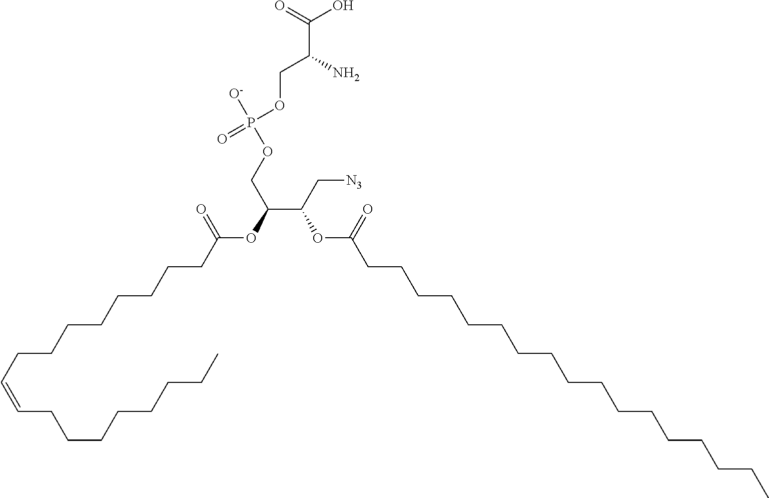 [SMILES: CCCCCCCC/C=C\CCCCCCCCC(=O)O[C@@H](COP(=O)([O-])OC[C@@H](N)C(=O)O)[C@H](CN=[N+]=[N-])OC(=O)CCCCCCCCCCCCCCCCC] |
| Compound Id | 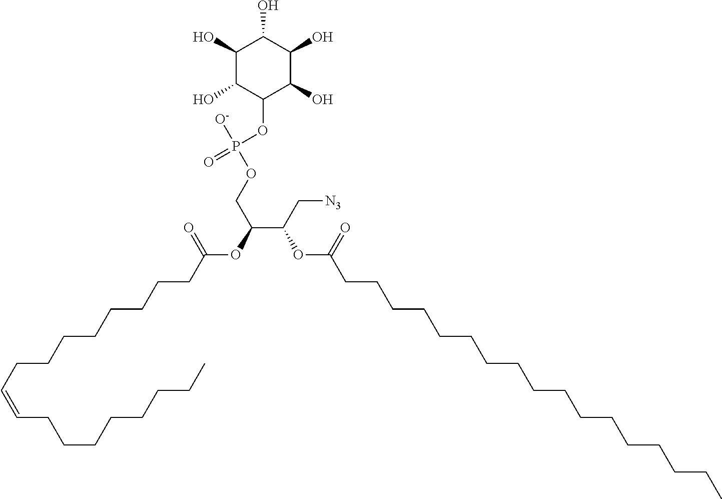 [SMILES: CCCCCCCC/C=C\CCCCCCCCC(=O)O[C@@H](COP(=O)([O-])OC1[C@H](O)[C@H](O)[C@@H](O)[C@H](O)[C@H]1O)[C@H](CN=[N+]=[N-])OC(=O)CCCCCCCCCCCCCCCCC] |
| Compound Ie | 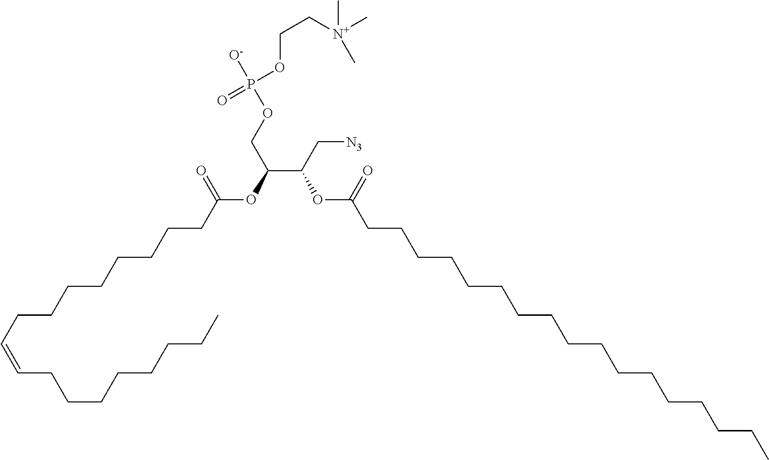 [SMILES: CCCCCCCC/C=C\CCCCCCCCC(=O)O[C@@H](COP(=O)([O-])OCC[N+](C)(C)C)[C@H](CN=[N+]=[N-])OC(=O)CCCCCCCCCCCCCCCCC] |

The second subject of the invention is a process for preparing a GPL of formula (I) as defined in the first subject of the invention, characterized in that it comprises at least the following steps:
i) a step of preparing a phosphotriester of formula (IV) comprising a diacetal function by functionalizing a diacetal derivative of (2S,3S)-butane-1,2,3,4-tetrol of formula (III) with a dialkyl halophosphate of formula (IV) in a basic medium, according to the following scheme:

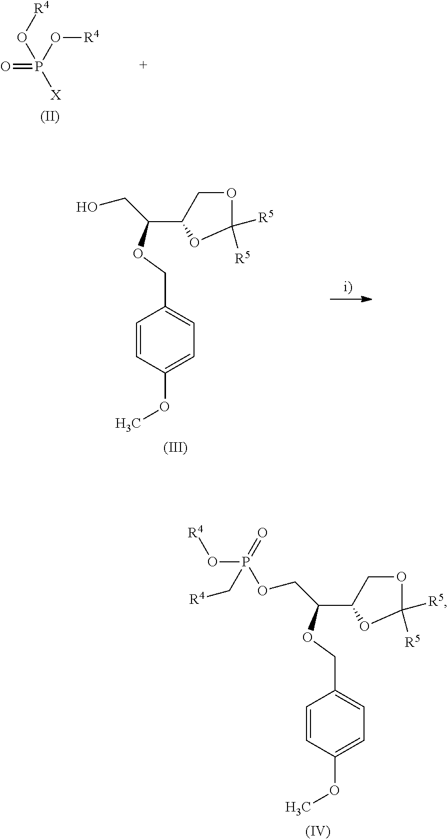

in which R⁴ represents a C₁-C₅ alkyl group or a benzyl group, and R⁵ represents a C₁-C₅ alkyl group,
ii) a step of deprotection in acidic medium of the diacetal function of the phosphotriester of formula (IV) to form a diol of formula (V) comprising a primary alcohol, according to the following scheme:

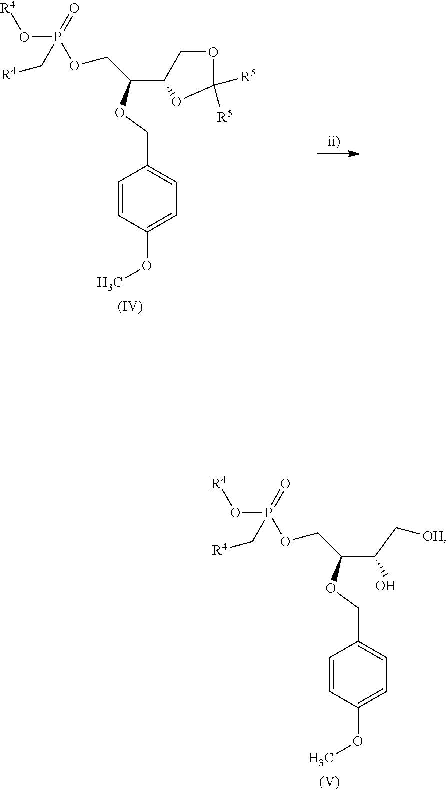

in which R⁴ is as defined in the invention,
iii) a step of selective functionalization of the primary alcohol of the diol of formula (V) to form a monoalcohol of formula (VI) comprising a primary alcohol modified with a group R⁶ (function OR⁶), according to the following scheme:

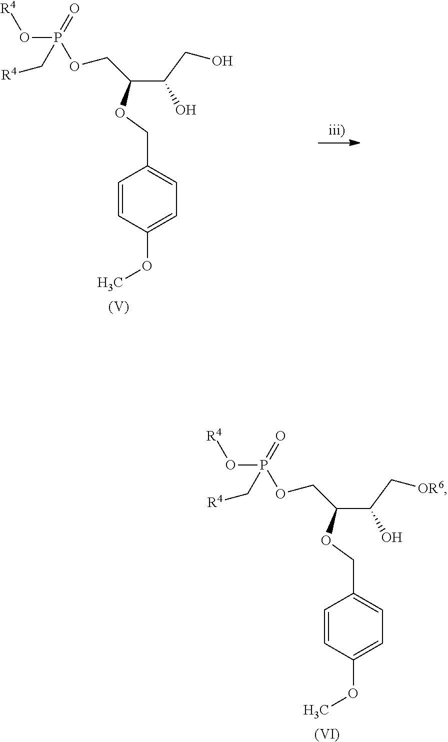

in which R⁴ is as defined in the invention and R⁶ is chosen from the following groups: tosyl, mesyl and triflate,
iv) a step of nucleophilic substitution of the function OR⁶ of the monoalcohol of formula (VI) to form an azide of formula (VII) comprising an alcohol function, according to the following scheme:

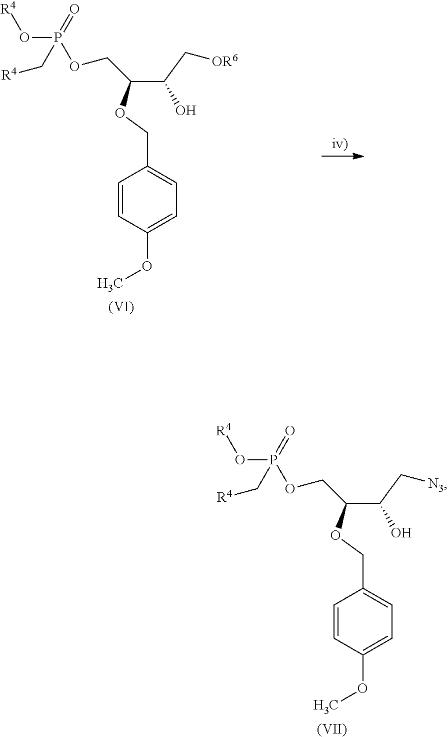

v) a step of esterification of the alcohol function of the azide of formula (VII) with a saturated fatty acid of formula (VIII) to form an azide of formula (IX) comprising a saturated fatty acid chain and an alcohol function protected with a para-methoxybenzyl (PMB) group, according to the following scheme:

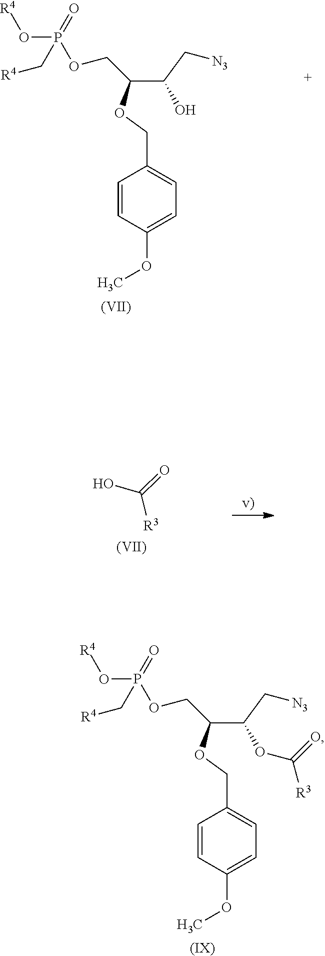

in which R³ and R⁴ are as defined in the invention,
vi) a step comprising a sub-step vi-1) of deprotection of the para-methoxybenzyl group of the azide of formula (IX) to form an azide of formula (X) comprising an alcohol function, followed by a sub-step vi-2) of esterification of the alcohol function of the azide of formula (X) with an unsaturated fatty acid of formula (XI), to form an azide of formula (XII) comprising a saturated fatty acid chain, an unsaturated fatty acid chain, and a phosphotriester function, according to the following scheme:

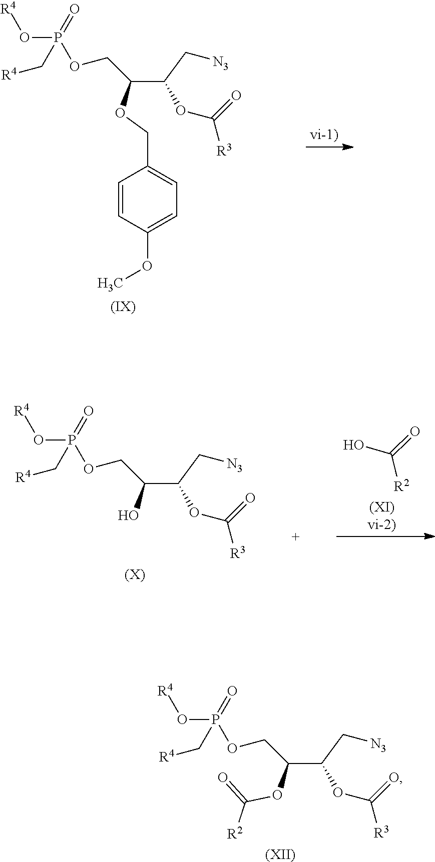

in which R² is as defined in the invention, and
vii) a step of deprotecting the groups R⁴ to form a compound of formula (I) in which OR¹═OH, optionally followed by a step of preparing a compound of formula (I) in which OR¹≠OH.

The process of the invention readily affords access to GPLs of formula (I) via a convergent synthetic route involving a synthetic intermediate of formula (VII) common to all GPLs of formula (I).

Consequently, the regio- and stereo-selective introduction of saturated and unsaturated fatty acid chains is performed as late as possible, i.e. during the two penultimate steps. This is all the more important for the introduction of the unsaturated fatty acid chain, since unsaturations may be sensitive to certain reaction conditions and/or may oxidize readily. The para-methoxybenzyl (PMB) group notably allows the alcohol function in the sn-2 position to be protected throughout the process of the invention, right up to the introduction of the unsaturated fatty acid chain. Moreover, deprotection of the phosphate group with the groups R⁴ is performed in a final step, so as to facilitate the purifications and/or to avoid the formation of byproducts during the preceding steps.

### Step i)

Step i) is a step of preparing a phosphotriester of formula (IV) comprising a diacetal function by functionalizing a diacetal derivative of (2S,3S)-butane-1,2,3,4-tetrol corresponding to formula (III) with a dialkyl halophosphate of formula (IV) in a basic medium.

In particular, the (2S,3S)-butane-1,2,3,4-tetrol diacetal derivative of formula (III) used in step i) already has the right stereochemistry at the sn-2 position to access the GPLs of formula (I). This stereochemistry is conserved throughout the process of the invention.

Step i) allows the introduction of a protected phosphate function in the sn-3 position of the diacetal derivative (III). This step i) is performed in a basic medium, for example in the presence of a base chosen from potassium tert-butoxide (tBuOK), NaH, NaNH₂ and KH.

Step i) is preferably performed at a temperature ranging from −20° C. to 50° C.

Step i) may be performed in a solvent such as dichloromethane, tetrahydrofuran (THF) or toluene.

The dialkyl halophosphate of formula (IV) is preferably chosen from dialkyl chlorophosphates such as dimethyl chlorophosphate.

R⁴ preferably represents a methyl group.

R⁵ preferably represents a methyl group.

### Step ii)

Step ii) is a step of deprotection in an acidic medium of the diacetal function of the phosphotriester of formula (IV) to form a diol of formula (V) comprising a primary alcohol function.

During this step ii), two alcohol functions are obtained, including a primary alcohol function in the sn-0 position.

This step ii) may be performed in a solvent chosen from lower (i.e. C₁-C₄) alcohols, such as methanol, ethanol or isopropanol.

Step ii) is performed in the presence of an acid chosen from hydrochloric acid (HCl), and any other suitable acid.

According to a preferred embodiment, step ii) is performed by acid methanolysis, in the presence of HCl.

Step ii) is preferably performed at room temperature (i.e. 18-25° C.).

Step iii)

Step iii) is a step of selective protection of the primary alcohol function of the diol of formula (V) to form a monoalcohol of formula (VI) comprising a primary alcohol function protected with a group R⁶.

Step iii) is preferably performed in the presence of a compound R⁶Y, Y being a halogen atom chosen from chlorine, bromine and iodine atoms.

Step iii) is preferably performed in the presence of a base, and optionally of a catalyst.

The base may be chosen from diisopropylethylamine (DIPEA), triethylamine (TEA), potassium carbonate (K₂CO₃), sodium hydroxide (NaOH) and potassium hydroxide (KOH).

The catalyst may be chosen from tin-based catalysts such as Bu₂SnO.

The group R⁶ is preferably a tosyl group.

Y is preferably a chlorine atom.

In a preferred embodiment, step iii) is performed in the presence of tosyl chloride, a base, and a tin-based catalyst.

Step iii) may be performed in an aprotic solvent such as toluene.

Step iii) is preferably performed at room temperature (i.e. 18-25° C.).

### Step iv)

Step iv) is a step of nucleophilic substitution of the primary alcohol function protected with a group R⁶ of the monoalcohol of formula (VI) to form an azide of formula (VII) comprising an alcohol function.

This step iv) allows the introduction of the azide function by means of displacement of the group —OR¹ by nucleophilic substitution.

Step iv) is preferably performed in the presence of an azide compound chosen from sodium azide, potassium azide, tetrabutylammonium iodide, diphenylphosphoryl azide and trimethylsilyl azide.

Step iv) is preferably performed at a temperature ranging from 0° C. to 100° C. Step iv) is generally performed in a polar solvent, which is preferably capable of dissolving the azide salts, such as dimethylformamide (DMF).

This azide of formula (VII) is a key intermediate compound in the synthesis of the synthetic GPLs of formula (I). By virtue of this, GPLs bearing various fatty acid chains in the sn-1 and sn-2 positions may be obtained in just three steps.

On conclusion of step iv), the crude reaction product may comprise the azide of formula (VII) as a mixture with the azide of formula (VII′) below:

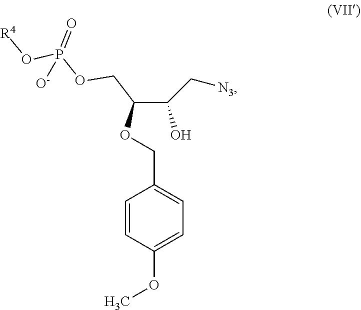

in which the phosphate triester has been partially deprotected (deprotection of a group R⁴).

The yield of this step iv) may be improved by treating the crude reaction product obtained on conclusion of step iv) with a diazomethane derivative such as trimethylsilyldiazomethane, 1-(mesitylene-2-sulfonyl)-3-nitro-1H-1,2,4-triazole, SiO₂Cl, CsF/MeI, or trimethyl orthoformate; and an alcohol R⁴OH, R⁴ being as defined in the invention, so as to yield the azide of formula (VII).

### Step v)

Step v) is a step of esterification of the alcohol function of the azide of formula (VII) with a saturated fatty acid of formula (VIII) to form an azide of formula (IX) comprising a saturated fatty acid chain of formula and an alcohol function protected with a para-methoxybenzyl (PMB) group.

This step v) may include a coupling agent, in particular chosen from carbodiimides such as N,N′-dicyclohexylcarbodiimide (DCC), N-(3-dimethylaminopropyl)-N′-ethylcarbodiimide (EDCl), N-cyclohexyl-N′-isopropylcarbodiimide (CIC), or N,N′-diisopropylcarbodiimide (DIC); and optionally a catalyst such as dimethylamidopyridine (DMAP) or pyridine.

Step v) is generally performed in a non-nucleophilic solvent such as dichloromethane, or chloroform.

Step v) is preferably performed at room temperature (i.e. 18-25° C.).

The fatty acid of formula (VIII) may be chosen from stearic acid, caproic acid, lauric acid, myristic acid, palmitic acid, arachidic acid, behenic acid, lignoceric acid, cerotic acid, montanic acid and melissic acid.

### Step vi)

Step vi) comprises a sub-step vi-1) of deprotection of the para-methoxybenzyl group of the azide of formula (IX) to form an azide of formula (X) comprising an alcohol function, followed by a sub-step vi-2) of esterification of the alcohol function of the azide of formula (X) with an unsaturated fatty acid of formula (XI), to form an azide of formula (XII) comprising a saturated fatty acid chain, an unsaturated fatty acid chain, and a phosphotriester function.

Sub-step vi-1) may be performed in the presence of a deprotecting agent chosen from 2,3-dichloro-5,6-dicyano-1,4-benzoquinone (DDQ), cerium ammonium nitrate (CAN), aluminum trichloride (AlCl₃) in the presence of ethanethiol (EtSH), tin tetrachloride (SnCl₄) in the presence of phenyl thiol (PhSH), or cerium trichloride (CeCl₃) in the presence of sodium iodide (NaI).

Sub-step vi-1) is preferably performed in a two-phase solvent mixture such as a mixture of dichloromethane and water.

Sub-step vi-1) is preferably performed at a temperature ranging from −20° C. to 50° C.

In a preferred embodiment, sub-step vi-1) is performed in the presence of 2,3-dichloro-5,6-dicyano-1,4-benzoquinone, and at a temperature ranging from −20° C. to 50° C.

On conclusion of sub-step vi-1), the alcohol of formula (X) obtained is employed directly in sub-step vi-2), in particular without a purification step and immediately. This thus allows its degradation to be avoided.

Sub-step vi-2) may include a coupling agent, in particular chosen from carbodiimides such as N,N′-dicyclohexylcarbodiimide (DCC), N-(3-dimethylaminopropyl)-N′-ethylcarbodiimide (EDCl), N-cyclohexyl-N′-isopropylcarbodiimide (CIC), or N,N′-diisopropylcarbodiimide (DIC); and optionally a catalyst such as dimethylamidopyridine (DMAP) or pyridine.

Sub-step vi-2) is generally performed in a non-nucleophilic solvent such as dichloromethane, or chloroform.

Sub-step vi-2) is preferably performed at a temperature ranging from −25° C. to 0° C.

The fatty acid of formula (XI) may be chosen from oleic acid, linoleic acid, lauroleic acid, myristoleic acid, palmitoleic acid, vaccenic acid and gadoleic acid, ketoleic acid, erucic acid, selacholeic acid, linolenic acid, eleostearic acid, arachidonic acid, cuplanodonic acid, elaidic acid and petroselinic acid.

The temperature ranges of sub-steps vi-1) and vi-2) and the implementation of sub-step vi-2) directly after sub-step vi-1) allow the deprotection of the PMB group to be promoted while at the same time avoiding the reaction of the ester borne by the azide of formula (X) with itself via an intramolecular transesterification mechanism which would then induce a loss of regioselectivity in the localization of the fatty acid chains which can be monitored by phosphorus NMR.

Step vii)

Step vii) consists in deprotecting the groups R⁴ to form a compound of formula (I) in which OR¹═OH, optionally followed by a step of preparing a compound of formula (I) in which OR¹≠OH.

The deprotection of the groups R⁴ may be performed in the presence of a reagent chosen from bromotrimethylsilane, butylamine, ammonia, trimethylamine, triethylamine, boron tribromide (BBr₃), pyridine, sodium iodide (NaI), and lithium cyanide (LiCN).

Step vii) is preferably performed in a solvent such as chloroform, DMF or toluene.

Step vii) is preferably performed at room temperature (i.e. 18-25° C.).

On conclusion of step vii), a GPL of formula (I) comprising a phosphate polar head (R¹=H) is obtained.

The (2S,3S)-butane-1,2,3,4-tetrol diacetal derivative of formula (III) as used in step i) may be prepared from a dialkyl-L-tartrate of formula (XIII) below:

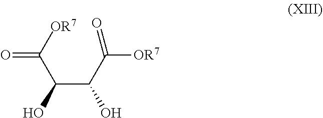

in which R⁷ is a C₁-C₅ alkyl group, preferably a methyl group.

In particular, the diacetal derivative (III) may be obtained by a method similar to that described by Namysio et al., J. Prakt. Chem., 1999, 341, 6, 557-561.

According to a particularly preferred embodiment of the invention, the diacetal derivative (III) is obtained according to the steps described in the following scheme:

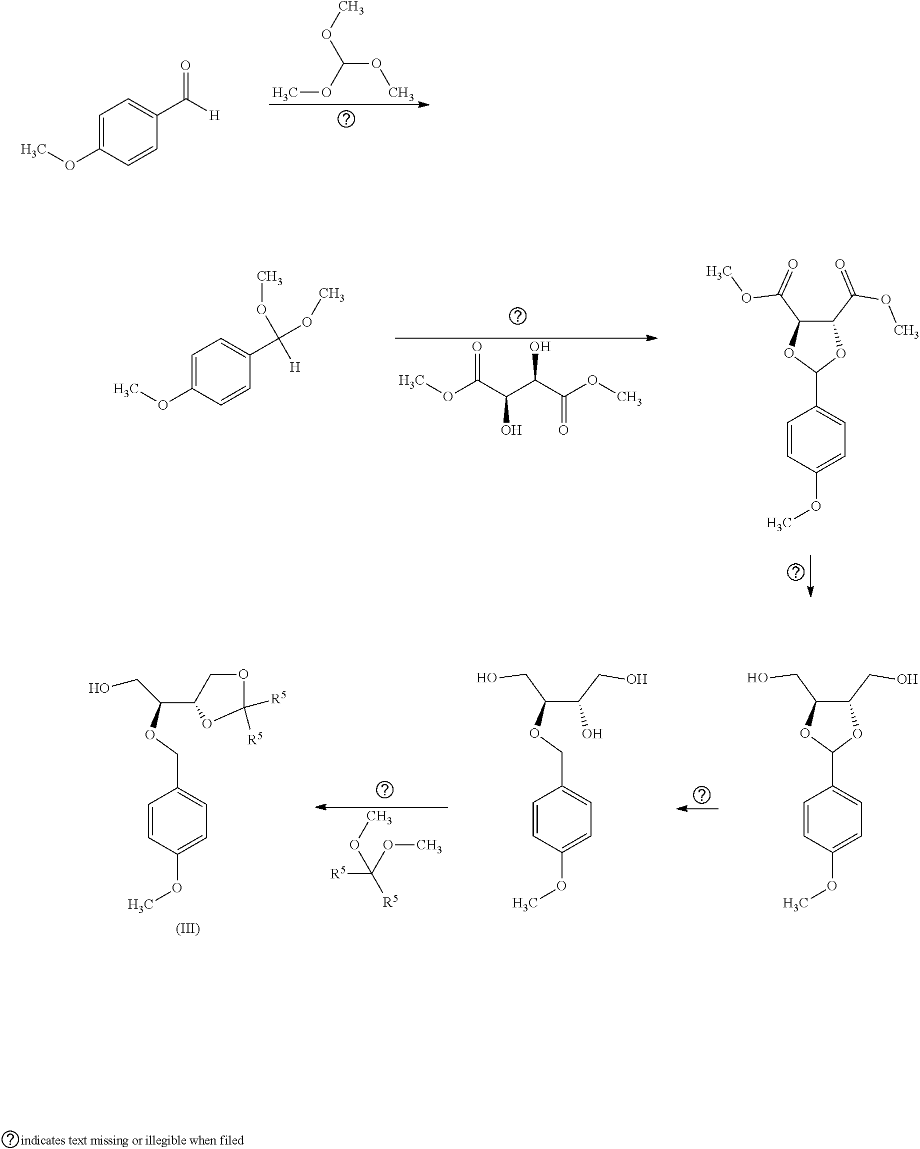

with R⁵ being as defined previously.

This method notably comprises the protection of dimethyl L-tartrate in the form of a diacetal, followed by two successive reductions of the esters and opening of the acetal to form a triol whose two adjacent alcohol functions are then protected with a dialkyl ketal.

The diacetal derivative (III) may also be obtained according to the steps described in the following scheme:

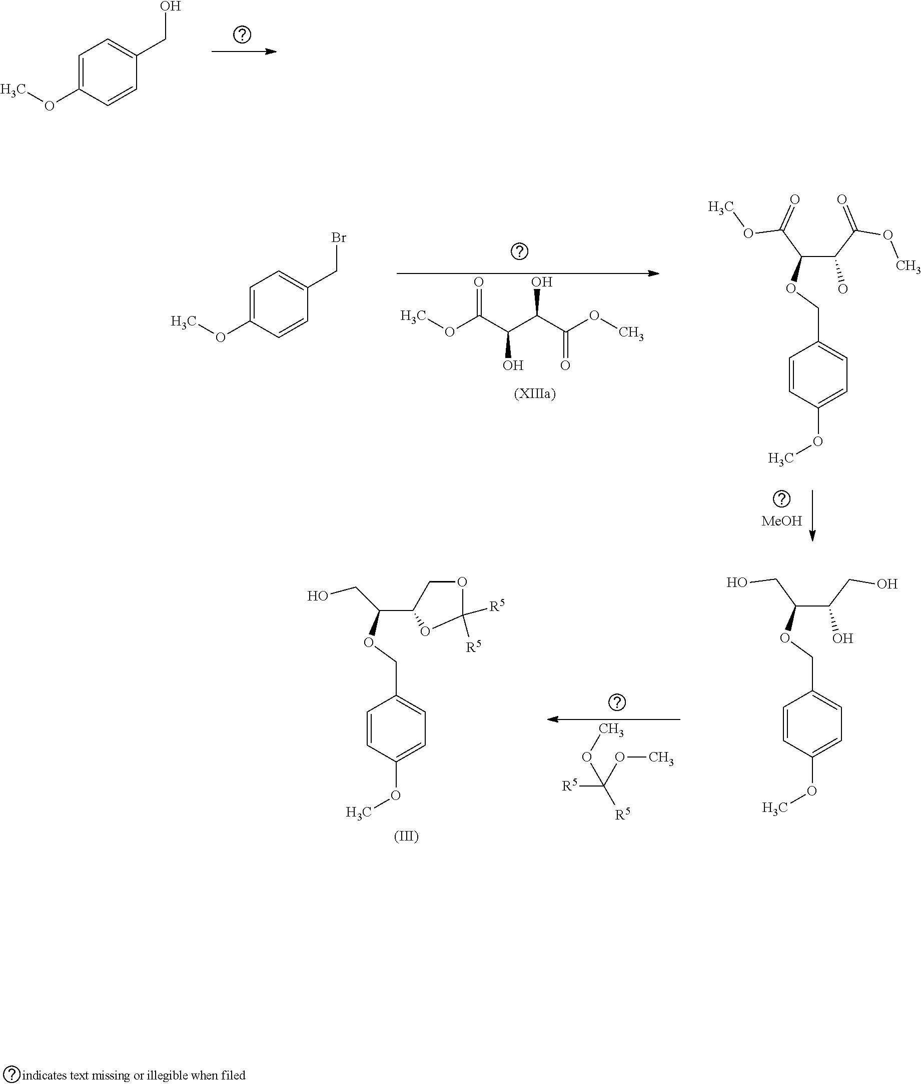

with R⁵ being as defined previously.

This method notably comprises the selective monoprotection of dimethyl-L-tartrate followed by two successive reductions of the esters and protection with a dialkyl ketal of the two adjacent alcohol functions.

The third subject of the invention is a modified glycerophospholipid corresponding to formula (XIV) below:

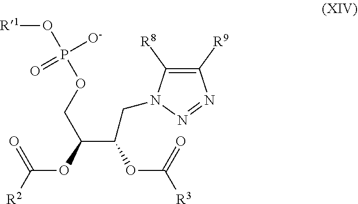

in which:
OR′¹ represents a group OR¹ as defined in the invention or a photoactivatable group,
R² and R³ are as defined in the first subject of the invention, and
R⁸ and R⁹, independently of each other, are chosen from a fluorophoric group, a photocrosslinkable group, a radiolabeling group and a hydrogen atom; or the groups R⁸ and R⁹ together form a group chosen from a fluorophoric group, a photocrosslinkable group and a radiolabeling group.

The modified GPLs of formula (XIV) conserve the structure and stereochemistry of natural GPLs.

The fluorophoric group may be chosen from nitrobenzoxadiazole (NBD)-based fluorophores, fluorophores bearing a xanthene unit, carborhodamines, cyanines, coumarins, and polycyclic aromatic hydrocarbons such as pyrenes or porphyrins.

Coumarins, nitrobenzoxadiazole (NBD)-based fluorophores and carborhodamines are preferred.

As examples of fluorophoric groups that may be used according to the invention, mention may be made of the fluorophores from the Alexa Fluor range sold by the company Molecular Probes.

The photocrosslinkable group as R⁸ and/or R⁹ may comprise (or consist of) an aryl azide group, a diazirine group, a diazo group, a benzophenone group, a benzophenone-based group, a tetrazole group or a quinoxalinone group.

The radiolabeling group may comprise (or consist of) fluorine-18, carbon-11, iodine-123, iodine-124, or a complex of gallium-67, indium-111m, technetium-99m or thallium-201.

Preferably, R⁸ is a hydrogen atom and R⁹ is a fluorophoric group, R⁹ is a hydrogen atom and R⁸ is a fluorophoric group, or the groups R⁸ and R⁹ together form a fluorophoric group.

The photoactivatable group as OR′¹ may comprise an aromatic group containing one or more nitro functions —NO₂ such as an ortho-nitrobenzyl or ortho-nitroindoline group, a coumarin group such as a coumarin-4-yl group, or a quinoline group.

The fourth subject of the invention is a process for preparing a modified glycerophospholipid of formula (XIV) as defined in the third subject of the invention, characterized in that it comprises at least one step A) of placing in contact a synthetic glycerophospholipid of formula (I) as defined in the first subject of the invention or obtained according to a process as defined in the second subject of the invention, with an alkyne corresponding to formula (XV) below:

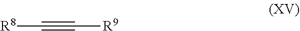

in which R⁸ and R⁹ are as defined in the invention.

Step A) may be performed by Huisgen cycloaddition, by Huisgen cycloaddition catalyzed with copper salts (CuAAC), or by Huisgen cycloaddition involving a constrained alkyne (SPAAC). Huisgen cycloaddition using a constrained alkyne (SPAAC) is preferred. This synthetic route notably circumvents the cytotoxicity of copper.

The alkyne of formula (XV) is preferably a constrained alkyne, in particular comprising a cyclooctyne group such as a difluorocyclooctyne group (DIFO), a difluorobenzocyclooctyne group (DIFBO), a dibenzocyclooctyne group (DIBO), a biarylazacyclooctynone group (BARAC) or a bicyclo[6.1.0]nonyne group (BCN).

By way of example of a constrained alkyne of formula (XV) and comprising a cyclooctyne group, mention may be made of compound (XVa) below comprising nitrobenzodiazole as fluorophore and a DIBO group as constrained alkyne:

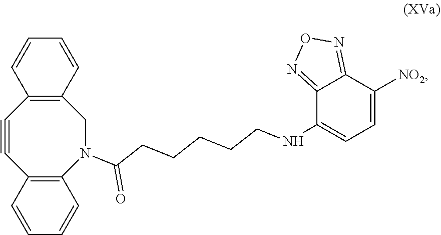 [SMILES: O=C(CCCCCNc1ccc([N+](=O)[O-])c2nonc12)N1Cc2ccccc2C#Cc2ccccc21]

or compound (XVa-2) below comprising a carborhodamine as fluorophore and a DIBO group as constrained alkyne:

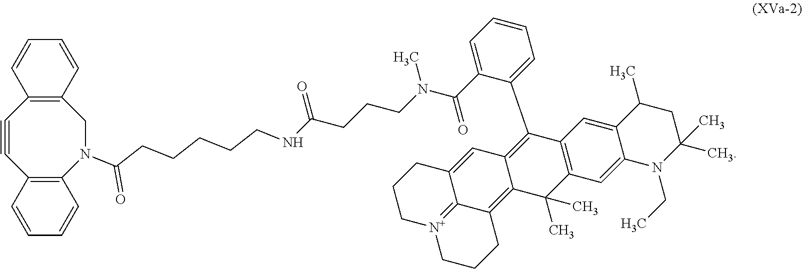 [SMILES: CCN1c2cc3c(cc2C(C)CC1(C)C)C(c1ccccc1C(=O)N(C)CCCC(=O)NCCCCCC(=O)N1Cc2ccccc2C#Cc2ccccc21)=c1cc2c4c(c1C3(C)C)CCC[N+]=4CCC2]

Step A) may be performed before or after placing the synthetic GPL (I) in contact with a biological medium, and preferably after placing the synthetic GPL (I) in contact with a biological medium.

According to a preferred embodiment of the invention, step A) is performed after placing the synthetic GPL (I) in contact with a biological medium. According to a particularly preferred embodiment of the invention, the process then also comprises, prior to step A), a step a) of placing the synthetic GPL (I) in contact with a biological medium, in particular in vitro, in vivo, ex vivo or in cellulo, and preferably in vitro or ex vivo.

Consequently, it is possible to address the synthetic GPL (I) in a cell, and then incorporate onto the glycerol backbone according to step A) different functional groups, which will notably depend on the envisaged application.

When R′¹ is a photoactivatable group, said group may be introduced before or after step A).

The process ensures the structure and stereochemistry of natural GPLs, of the unmodified polar head, optionally caged by a photolabile group to prevent their metabolization prior to biological study, and of the intact, correctly positioned fatty acid chains. Furthermore, a single synthetic GPL (I) allows access to several types of modified GPL (XIV) and thus access to a wide range of applications for a single synthetic GPL (I) prepared. This thus allows modified GPLs (XIV) to be obtained which are structurally more similar to natural GPLs, and to offer great flexibility and diversity in the choice of tools for biological studies.

The fifth subject of the invention is the use of a synthetic glycerophospholipid of formula (I) as defined in the first subject of the invention or obtained according to a process as defined in the second subject of the invention; or of a modified glycerophospholipid (XIV) as defined in the third subject of the invention or obtained according to a process as defined in the fourth subject of the invention, as a molecular tool, in particular in the field of biology, or as a biological probe, in particular for biological studies.

These synthetic glycerophospholipids of formula (I) and modified glycerophospholipids of formula (XIV) may, for example, be used to understand the cellular processes and/or biological functions of natural GPLs depending on the types of polar head and/or fatty acid chains present in said GPLs.

In particular, it has been shown that the synthetic GPLs of formula (I) may interact in the same way as natural GPLs with mobilizing proteins such as the green-fluorescent Spo20p-GFP (see Example 3 of the application hereinbelow), or chromogranin A (or CgA).

In particular, it has been shown that modified GPLs of formula (XIV) may interact just like natural GPLs with mobilizing proteins such as chromogranin A (or CgA).

GPLs (I) and (XIV) may be used in the form of a kit for the assay and/or localization of biomolecules, such as proteins.

The sixth subject of the invention is the use of a synthetic glycerophospholipid of formula (I) as defined in the first subject of the invention or obtained according to a process as defined in the second subject of the invention; or a modified glycerophospholipid of formula (XIV) as defined in the third subject of the invention or obtained according to a process as defined in the fourth subject of the invention, as a diagnostic tool, and preferably as an in vitro or ex vivo diagnostic tool.

in vitro diagnosis is performed outside the human body on a biological medium. ex vivo diagnosis is performed outside the human body on a biological medium that has been pretreated prior to use.

In other words, the invention relates to a process involving placing in contact a synthetic glycerophospholipid of formula (I) as defined in the first subject of the invention or obtained according to a process as defined in the second subject of the invention; or a modified glycerophospholipid of formula (XIV) as defined in the third subject of the invention or obtained according to a process as defined in the fourth subject of the invention, with a biological medium for diagnostic purposes, and in particular for localizing and/or identifying the various proteins which interact therewith.

The seventh subject of the invention is a synthetic glycerophospholipid of formula (I) as defined in the first subject of the invention or obtained according to a process as defined in the second subject of the invention; or a modified glycerophospholipid of formula (XIV) as defined in the third subject of the invention or obtained according to a process as defined in the fourth subject of the invention, for its medical use.

In other words, the invention relates to the use of a synthetic glycerophospholipid of formula (I) as defined in the first subject of the invention or obtained according to a process as defined in the second subject of the invention; or a modified glycerophospholipid of formula (XIV) as defined in the third subject of the invention or obtained according to a process as defined in the fourth subject of the invention, for obtaining or preparing a medicament intended for therapeutic use (i.e. for the production or preparation of a medicament for therapeutic treatment). The invention also relates to a treatment method comprising the administration of a synthetic glycerophospholipid of formula (I) as defined in the first subject of the invention or obtained according to a process as defined in the second subject of the invention; or a modified glycerophospholipid of formula (XIV) as defined in the third subject of the invention or obtained according to a process as defined in the fourth subject of the invention, notably in a therapeutic amount, to a patient. In particular, GPLs (I) and (XIV) comprising three successive unsaturations on the unsaturated fatty acid chain may be used to improve the secretion of neurotransmitters by neurosecretory cells and/or treat diseases of the nervous system, in particular linked to normal or pathological aging, or linked to mental retardation (Fragile X, Coffin Lowry Syndrome).

### BRIEF DESCRIPTION OF THE DRAWINGS

The attached drawings illustrate the invention:

FIG. 1 represents confocal microscopy images of media comprising different GPLs incubated with PC12 cells expressing Spo20p-GFP.

FIG. 2 represents the percentage colocalization of Spo20p-GFP protein and different GPLs in the plasma membrane.

FIG. 3 represents light microscopy images of liposomes containing different GPLs.

FIG. 4 shows confocal microscopy images of liposomes containing different GPLs.

FIG. 5 shows confocal microscopy images of liposomes containing different GPLs incubated with a GgA-AF633 protein.

Other features, variants and advantages of the use of the phytotoxic composition or of the process according to the invention will emerge more clearly on reading the implementation examples that follow, which are given as nonlimiting illustrations of the invention.

### EXAMPLES

The reagents are from commercial sources (Sigma-Aldrich, Acros, Alfa-Aesar) as are the solvents (Sigma-Aldrich, Acros), and were used as received from the manufacturers, without specific treatment unless otherwise indicated.

The nuclear magnetic resonance (NMR) experiments were performed using a spectrometer sold under the trade name Avance DPX300 by Brüker, operating at 293 K with a frequency of 300.13 MHz for proton (¹H) NMR, 121.442 MHz for phosphorus (³¹P) NMR and 75.48 MHz for carbon (¹³C) NMR. The chemical shifts are expressed in ppm (parts per million) by comparison with an internal reference, tetramethylsilane (TMS, δ=0 ppm), and internal calibrations are performed using a residual solvent signal. The proton NMR coupling constants are reported in hertz and the multiplicities are specified (s: singlet; d: doublet; t: triplet; m: multiplet; dd: doublet of doublets).

Electrospray ionization mass spectrometry (ESI-MS) data were acquired using a spectrometer sold under the trade name LCT Premier XE by Waters Acquity. Exact mass measurements (HRMS) were performed using a Synapt G2 HDMS system and an electrospray source equipped with a Lockspray® system. The experiments were performed with a positively charged Waters Acquity BEH reference C18 column having the following specificities: 1.7 μm; 2.1-50 mm. The gradient used was as follows: 98%/2% H₂O/ACN to 100% ACN in 4 min and 1.3 min at 100% ACN with a flow rate of 0.25 ml·min⁻¹. 0.5 ml is injected, and the source temperature is set at 120° C. and the desolvation temperature at 300° C., with voltages of 2200 V for the detector and 3000 V for the capillary.

Analyses by ultra-high performance liquid chromatography (UHPLC) were recorded on a machine sold under the trade name Ultimate 3000 by Thermo Fisher. Experiments were performed on a Hypersil GOLD C18 reference column having the following specificities: 3 μm; 2.1-50 mm. The gradient used was as follows: 95%/5% ACN/H₂O to 100% H₂O in 6 min with a flow rate of 0.6 ml·min⁻¹ and a pressure of 450 bar. 10 μL are injected and the oven temperature is set at 25° C. The products were analyzed with a UV detector at 254 nm and 270 nm.

Elemental analyses were recorded on a machine sold under the trade name Flash 2000 by Thermo Fisher—EAGER 300. The determination of the general formula of an organic compound by the mass percentage of each of the elements (C, H, N, S) present in said compound is obtained by integrating the chromatographic peaks of each of the elements. The areas thus integrated are pointed at a calibration line so as to determine the concentration of each of the elements. The results are provided with an absolute accuracy of 0.4%.

Each sample is precisely weighed in a tin basket. The standard used for the calibration range is 2,5-bis(5-tert-butylbenzoxazol-2-yl)thiophene (BBOT) with the following composition: % C: 72.53; % H: 6.09; % N: 6.51; % S: 7.44.

The infrared spectra were acquired with a spectrophotometer sold under the trade name Perkin Elmer 100 FTIR (attenuated total reflectance or ATR technique using a diamond crystal). The products are analyzed directly on the diamond. The absorption bands are expressed in cm⁻¹.

### Example 1: Synthesis of Compound Ia

### Preparation of the GPL precursor (S)-2-[(S)-2,2-dimethyl-1,3-dioxolan-4-yl]-2-(4-methoxybenzyloxy)ethan-1-ol

(S)-2-[(S)-2,2-Dimethyl-1,3-dioxolan-4-yl]-2-(4-methoxybenzyloxy)ethan-1-ol was prepared according to the five steps illustrated in the following scheme:

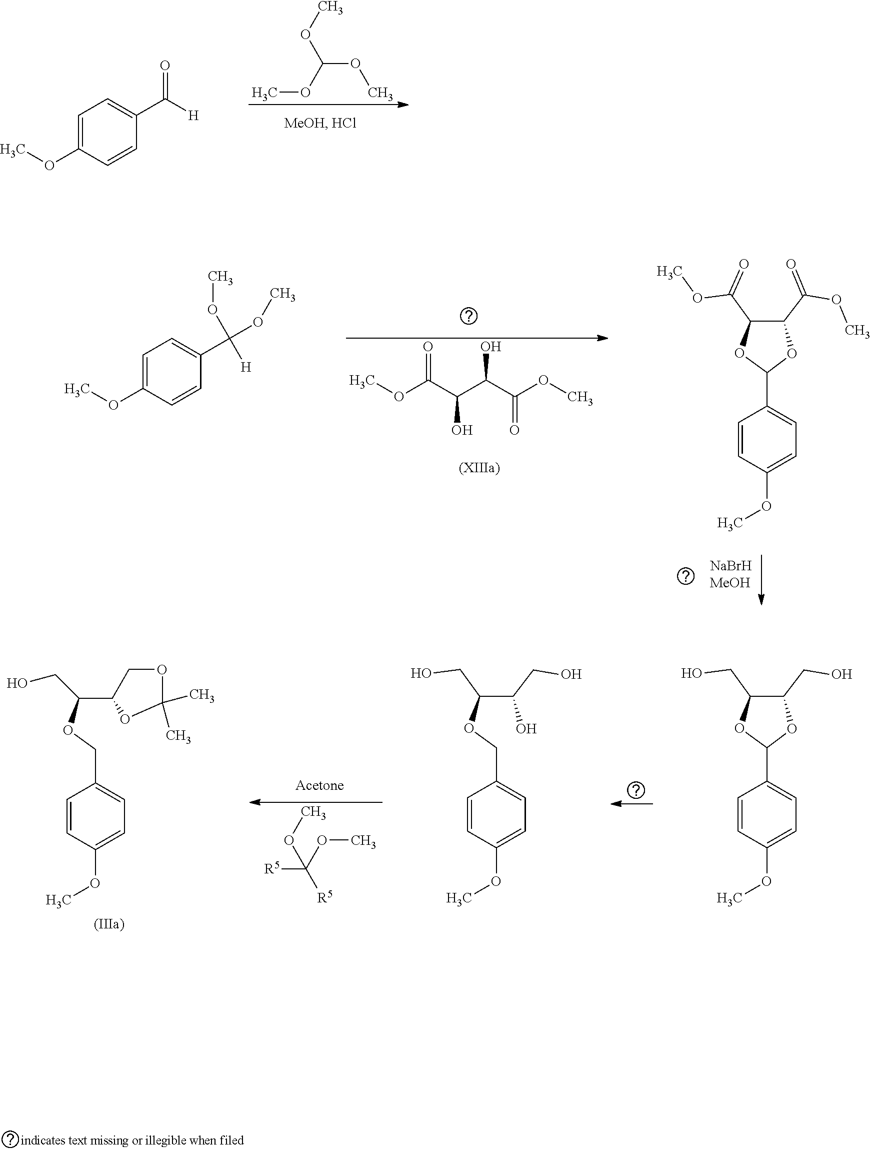

### First Step:

The first step is a reaction for converting the aldehyde into a diacetal.

78.5 g of p-methoxybenzaldehyde (580 mmol, 70 ml) and 61.5 g of trimethyl orthoformate (580 mmol, 68.1 ml, 1 equiv.) are stirred vigorously for 5 min in 25 ml of methanol (MeOH). Next, four drops of 37% HCl are added, the translucent orange mixture turns translucent pink and the solution is stirred vigorously overnight. Next, 450 mg of K₂CO₃ are added and the resulting mixture is stirred vigorously for 1 h. The color of the mixture changes from translucent pink to translucent yellow. 300 ml petroleum ether are then added and a cloudy, glossy white suspension is formed. The resulting mixture is filtered and the solvents are evaporated off under reduced pressure. The crude product is distilled under vacuum (5 mbar, boiling point=97° C.) so as to obtain 1-(dimethoxymethyl)-4-methoxybenzene in the form of a colorless liquid in 93% yield (97.7 g, 536 mmol).

Formula: C₁₀H₁₄O₃, Molar mass=182.22 g·mol⁻¹.

### Second Step:

The second step is a step of protecting dimethyl L-tartrate in the form of an acetal.

40 g of dimethyl L-tartrate (225 mmol, compound (XIIIa)) and 46 g of 1-(dimethoxymethyl)-4-methoxybenzene as prepared in the preceding step (337.5 mmol, 64.5 ml, 1.5 equiv.) are added to 500 ml of dry toluene under an inert atmosphere. The solution is stirred vigorously, 400 mg of para-toluenesulfonic acid (PTSA) (2.3 mmol, 0.01 equiv.) are added, and the methanol formed during the reaction is evaporated off by distillation. The color of the solution changes from translucent violet-pink to orange, and the distillation process is stopped once the distillate has reached a temperature of 110° C. The crude mixture is cooled to room temperature and 250 ml of dichloromethane (CH₂Cl₂ or DCM) are added, then the reaction is stopped with an excess of K₂CO₃. The crude mixture is stirred for a further hour to obtain a yellow colored mixture. The crude mixture is filtered and the solvents are evaporated off under reduced pressure to obtain a pale yellow solid, which is purified by chromatography on silica gel (eluent: petroleum ether+0.1% triethylamine (Et₃N)/ethyl acetate (EtOAc)). This purification step may be replaced by washing the pale yellow solid with cyclohexane. In both cases, a white solid is obtained in 90% yield corresponding to (4R,5R)-dimethyl-2-(4-methoxyphenyl)-1,3-dioxolane-4,5-dicarboxylate (60 g, 202.5 mmol).

Formula: C₁₄H₁₆O₇, Molar mass=296.27 g·mol⁻¹.

¹H NMR (300.13 MHz, CDCl₃), δ (ppm): 3.81, 3.84 and 3.87 (9H, OCH₃); 4.84 (d, J=4.0 Hz, 1H, CH(OR)); 4.95 (d, J=4.0 Hz, 1H, CH(OR)); 6.09 (s, 1H, CH(OR)₂); 6.92 (d, J=8.8 Hz, 2H, aromatic CH); 7.51 (d, J=8.7 Hz, 2H, aromatic CH).

¹³C NMR (75.49 MHz, CDCl₃), δ (ppm): 52.6, 52.7 and 55.1 (3C, OCH₃); 76.9, 77.1 (CH(OR)); 106.5 (CH(OR)₂); 113.6, 127.2, 128.5 and 160.8 (6C, aromatic C); 169.4 and 169.9 (2C, CO₂R).

### Third Step:

The third step is a step of reducing the ester functions to alcohol functions.

46 g (160 mmol) of the diester as prepared in the preceding step are dissolved in 750 ml of methanol under an inert atmosphere. The resulting mixture is stirred at 0° C. and 18.2 g (480 mmol, 3 equiv.) of NaBH₄ are added portionwise. The resulting mixture is stirred for 3 h at room temperature. The solvent is then evaporated off under reduced pressure. 500 ml of ethyl acetate are added and the resulting organic solution is washed with 200 ml of brine. The aqueous phase is extracted with 500 ml of ethyl acetate. The organic phases are combined, dried over MgSO₄ and filtered, and the solvent is evaporated off under reduced pressure. The product corresponding to (4S,5S)-2-(4-methoxyphenyl)-1,3-dioxolane-4,5-diyl)dimethanol is obtained in the form of a white solid without purification in 98% yield (23.1 g, 96 mmol).

Formula: C₁₂H₁₆O₅, Molar mass=240.25 g·mol⁻¹.

¹H NMR (300.13 MHz, CDCl₃), δ (ppm): 2.02 (bs, 2H, OH); 3.82 (s, 3H, OCH₃); 3.90 (4H, CH₂OH); 4.16 (m, 2H, CH(OR)); 5.95 (s, 1H, CH(OR)₂); 6.92 (d, J=8.8 Hz, 2H, aromatic CH); 7.51 (d, J=8.7 Hz, 2H, aromatic CH).

¹³C NMR (75.49 MHz, CDCl₃), δ (ppm): 55.4 (OCH₃); 62.4 and 62.5 (CH₂OH); 78.6 and 79.4 (CH(OR)); 103.8 (CH(OR)₂); 113.9, 128.1, 129.4 and 160.7 (6C, aromatic C).

### Fourth Step:

The fourth step is a step of opening of the acetal to form a triol in which the sn-1 position is freed while the sn-2 position is still protected.

30 g (125 mmol) of the diol prepared in the preceding step are dissolved in 750 ml of dry tetrahydrofuran (THF) under an inert atmosphere. The resulting mixture is cooled to 0° C. and 83.1 ml (291.2 mmol, 7 equiv.) of BH₃·Me₂S are added dropwise at 0° C. The resulting mixture is stirred for 30 min at room temperature and then for 3 h at 75° C. The resulting mixture is cooled to 0° C. and the excess borane is cautiously hydrolyzed with methanol. The mixture obtained is stirred for 30 min at room temperature and the solvents are evaporated off under reduced pressure. The residue is treated twice more with 250 ml of methanol and evaporated under reduced pressure. The crude solid obtained is extracted twice with CH₂Cl₂ and the insoluble white solid containing the boron salts is filtered off. The organic phases are recovered, evaporated and the resulting product purified by chromatography on silica gel (eluent: EtOAc/MeOH+0.1% Et₃N). This purification step may be replaced with recrystallization from toluene. In both cases, the product corresponding to (2S,3S)-3-(4-methoxybenzyloxy)butane-1,2,4-triol is obtained in the form of a white solid in 58% yield (17.5 g, 72.28 mmol).

Formula: C₁₂H₁₈O₅, Molar mass=242.27 g·mol⁻¹.

¹H NMR (300.13 MHz, CDCl₃), δ (ppm): 2.42-2.77 (3H, OH); 3.54-3.59 (m, 3H, CH(OR), CH₂); 3.68-3.89 (m, 3H, CH₂OH, CH(OH)); 3.76 (s, 3H, OCH₃); 4.52-4.65 (2d, J=11.3 Hz, 2H, benzyl CH₂); 6.90 (d, J=8.7 Hz, 2H, aromatic CH); 7.27 (d, J=8.7 Hz, 2H, aromatic CH).

¹³C NMR (75.49 MHz, CDCl₃), δ (ppm): 55.4 (OCH₃); 60.8 and 63.4 (2C, CH₂OH); 72.1 (CH(OH)); 72.3 (benzyl CH₂); 79.0 (CH(OR)); 114.1, 129.6, 129.8 and 159.6 (6C, aromatic C).

### Fifth Step:

The fifth step is a reaction to protect the two alcohol functions in the form of a dimethyl acetal.

14 g of triol as prepared in the preceding step (58.6 mmol) are dissolved in dry acetone (145 ml) under an inert atmosphere. 21.6 ml (175.8 mmol, 3 equiv.) of dimethoxypropane and 500 mg (2.9 mmol, 5 mol %) of para-toluenesulfonic acid (PTSA) are then added and the resulting mixture is stirred for 1 h. The color of the mixture changes from translucent to reddish-brown. 460 ml (3.3 mmol) of triethylamine are added and the color changes to translucent yellow. The solvents are evaporated off under reduced pressure and the crude product is purified by chromatography on silica gel (eluent: petroleum ether+0.1% triethylamine/EtOAc). The compound obtained (IIIa) corresponding to (S)-2-[(S)-2,2-dimethyl-1,3-dioxolan-4-yl]-2-(4-methoxybenzyloxy)ethan-1-ol is obtained in the form of a translucent pale yellow oil in 70% yield (11.53 g, 40.9 mmol).

Formula: C₁₅H₂₂O₅, Molar mass=282.27 g·mol⁻¹.

¹H NMR (300.13 MHz, CDCl₃), δ (ppm): 1.36 and 1.42 (3H each, CH₃); 2.19 (1H each, OH); 3.55 (m, 2H, CH₂(OH); 3.65 (m, 1H, CH(OPMB)); 3.70 (m, 1H, dioxolane CH₂(OR)); 3.80 (s, 3H, OCH₃); 4.00 (dd, J=8.3 Hz; 6.4 Hz, 1H, dioxolane CH₂(OR)); 4.28 (dt, J=8.3 Hz; 6.4 Hz, 1H, dioxolane CH(OR)); 4.59-4.72 (2d, J=11.5 Hz, 2H, benzyl CH₂); 6.89 (d, J=8.6 Hz, 2H, aromatic CH); 7.27 (d, J=8.6 Hz, 2H, aromatic CH).

¹³C NMR (75.49 MHz, CDCl₃), δ (ppm): 25.4 and 26.5 (CH₃); 55.4 (OCH₃); 61.8 (CH₂OH); 65.7 (dioxolane CH₂); 72.6 (benzyl CH₂); 76.8 (dioxolane CH) 78.9 (CH(OPMB)); 109.6 (dioxolane C(OR)₂(Me)₂); 114.1, 129.6, 129.8 and 159.6 (6C, aromatic C).

HRMS (ESI+) m/z calcd. For [C₁₅H₂₂O₅][M+NH₄]⁺: 300.1805; found: 300.1815.

IR (v cm⁻¹): 826 (v p-disubst. benzene); 1032 (v CH₂OH); 1110-1140 (v alkyl ethers); 1247 (v C═C—O—C); 1370 (v C—(CH₃)₂); 1375-1385 (v C—CH₃); 1590-1615 (v aryl); 2815-2835 (v O—CH₃)

### Preparation of GPL (Ia)

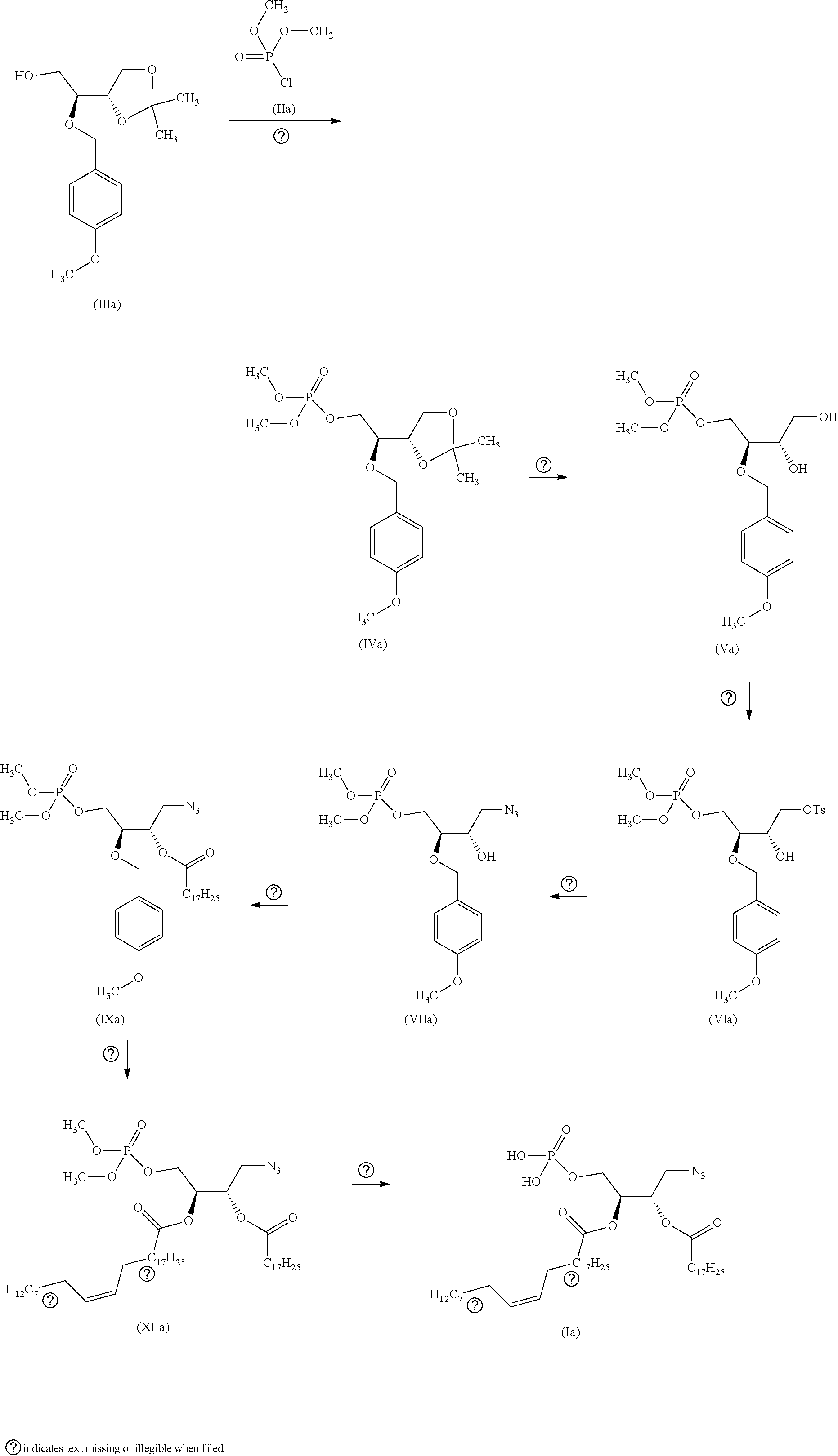

### First Step:

The first step is to functionalize the alcohol function in the sn-3 position of the diacetal (IIIa) to form a phosphotriester (IVa).

6 g (21.24 mmol) of the diacetal (IIIa) as prepared in the preceding step are dissolved in 90 ml of dry DCM under an inert atmosphere. The resulting mixture is cooled to 0° C. 2.98 g (26.58 mmol, 1.25 equiv.) of freshly sublimed potassium tert-butoxide are then added. The resulting mixture is stirred and the color changes to yellow. 2.86 ml (26.58 mmol, 1.2 equiv.) of freshly distilled dimethyl chlorophosphate (IIa) are added dropwise at 0° C., and the resulting mixture is stirred for 4 h at 0° C. 72 ml of saturated NaHCO₃ solution are then added and the resulting mixture is stirred for 1 h. The organic phase is separated out and the aqueous phase is extracted twice with 180 ml of DCM. The organic phases are combined and washed twice with 60 mL of saturated NaHCO₃ solution, dried, and the solvents are evaporated off under reduced pressure. The crude product is purified by chromatography on silica gel (eluent: petroleum ether+0.1% triethylamine/EtOAc). Compound (IVa) corresponding to [(S)-2-((S)-2,2-dimethyl-1,3-dioxolan-4-yl)-2-((4-methoxybenzyl)oxy]ethyl dimethyl phosphate is obtained in the form of a translucent pale yellow oil in 60% yield (4.93 g, 12.6 mmol).

Formula: C₁₇H₂₇O₈P, Molar mass=390.37 g·mol⁻¹.

¹H NMR (300.13 MHz, CDCl₃), δ (ppm): 1.34 and 1.41 (3H each, CH₃); 3.65 (m, 1H, dioxolane CH(OR)); 3.67 (m, 1H, dioxolane CH₂(OR)); 3.74 and 3.78 (s, 3H each, P—OCH₃); 3.80 (s, 3H, benzyl-OCH₃); 3.95-4.05 (dd, J=8.4 Hz; 6.6 Hz, 1H, dioxolane CH₂(OR)); 4.14-4.27 (m, 3H, CH₂ OP(O)(OCH₃)₂, CH(OPMB)); 4.59-4.72 (2d, J=11.3 Hz, 2H, benzyl CH₂); 6.87 (d, J=8.7 Hz, 2H, aromatic CH); 7.28 (d, J=8.6 Hz, 2H, aromatic CH).

³¹P NMR (121.442 MHz, CDCl₃), δ (ppm): 1.27 (P(O)(OMe)₂)

¹³C NMR (75.49 MHz, CDCl₃), δ (ppm): 25.4 and 26.5 (CH₃); 54.5 and 54.6 (2d, J=6.0 Hz, —P(O)(OCH₃)₂); 55.4 (benzyl-OCH₃); 65.5 (dioxolane CH₂); 67.0 (d, J=6.0 Hz, CH₂—OP(O)(OCH₃)₂); 72.8 (benzyl CH₂); 75.7 (CH(OPMB)); 77.1 (dioxolane CH); 109.6 (dioxolane C(OR)₂(Me)₂); 113.9, 129.7, 130.1 and 159.5 (6C, aromatic C).

HRMS (ESI+) m/z calcd. For [C₁₇H₂₇O₈P] [M+H]⁺: 391.1522; found: 391.1534.

IR (v cm⁻¹): 820 (v p-disubst. benzene); 1025 (v P—O—(C)); 1110-1140 (v alkyl ethers); 1214 (v C═C—O—C); 1247.13 (v P═O); 1360-1370 (v C—CH₃)₂)); 1375-1385 (v C—CH₃); 1590-1615 (v aryl); 2815-2835 (v O—CH₃); 3015 (v=C—H aryl)

### Second Step:

The second step is a step of deprotection of the alcohol functions in positions sn-1 and sn-0.

3.7 g (9.5 mmol) of the diacetal (IVa) as prepared in the preceding step are dissolved in 100 ml of dry methanol. 1.85 ml of 1M HCl are added dropwise and the resulting mixture is stirred for 8 h at room temperature. 350 mg of solid NaHCO₃ are added and the resulting mixture is stirred for 15 min. The solvent is evaporated off under reduced pressure. 150 ml of DCM are added and the mixture is filtered. The solvent is evaporated off under reduced pressure and a translucent oil is obtained. The crude product is purified by chromatography on silica gel (eluent: EtOAc/MeOH). Product (Va) corresponding to (2S,3S)-3,4-dihydroxy-2-((4-methoxybenzyl)oxy)butyl dimethyl phosphate is obtained in the form of a translucent pale yellow oil in 82% yield (2.8 g, 8.0 mmol).

Formula: C₁₄H₂₃O₈P, Molar mass=350.30 g·mol⁻¹.

¹H NMR (300.13 MHz, CDCl₃), δ (ppm): 2.37 (2H, OH); 3.65 (2H, CH₂OH); 3.70 (1H, CH(OPMB)); 3.73 (1H, CH(OH)); 3.75 and 3.79 (s, 3H each, P—OCH₃); 3.81 (s, 3H, benzyl OCH₃); 4.13-4.30 (2m, 2H, CH₂OP(O)(OCH₃)₂); 4.48-4.72 (2d, J=11.3 Hz, 2H, benzyl CH₂); 6.88 (d, J=8.7 Hz, 2H, aromatic CH); 7.27 (d, J=8.6 Hz, 2H, aromatic CH).

³¹P NMR (121.442 MHz, CDCl₃), δ (ppm): 1.51 (P(O)(OMe)₂).

¹³C NMR (75.49 MHz, CDCl₃), δ (ppm): 54.5 and 54.6 (2d, J=6.0 Hz, —P(O)(OCH₃)₂); 55.4 (benzyl OCH₃); 63.4 (CH₂OH); 66.2 (d, J=6.0 Hz, CH₂—OP(O)(OCH₃)₂); 70.9 (CH(OH)); 72.9 (benzyl CH₂); 77.4 (CH(OPMB)); 114.0, 129.6, 129.9 and 159.5 (6C, aromatic C).

HRMS (ESI+) m/z calcd. For [C₁₄H₂₃O₈P] [M+H]⁺: 351.1209; found: 351.1223.

IR (v cm⁻¹): 820 (v p-disubst. benzene); 1031.76 (v P—O—(C)); 1110-1140 (v alkyl ethers); 1180.02 (v C═C—O—C); 1248.02 (v P═O); 1375-1385 (v C—CH₃); 1462.86 (v alkyls CH₂); 1590-1615 (v aryl); 2815-2835 (v O—CH₃); 2957.67 (v alkyls, CH₃); 3010 (v=C—H aryl); 3403.97 (v 0-H)

### Third Step:

The third step is a step of selective functionalization of the primary alcohol function.

3.6 g (10.27 mmol) of diol (Va) as prepared in the preceding step are dissolved in 15 ml of dry toluene under an inert atmosphere. 12.8 mg (51.37 μmol, 0.5 mol %) of Bu₂SnO are added and the resulting mixture is stirred for 1 h. 1.9 ml (12.33 mmol, 1.2 equiv.) of diisopropylethylamine (DIPEA) are then added and the resulting mixture stirred for 5 min. Next, 2.06 g (10.79 mmol, 1.1 equiv.) of tosyl chloride (TsCl) are added and the resulting mixture is stirred for 4 h at room temperature. A yellow aqueous phase appears. 1M HCl solution is added with vigorous stirring until the pH of the aqueous phase is about 1-2. The organic phase is separated out and the aqueous phase is extracted twice with 40 ml of DCM. The organic phases are combined and dried over magnesium sulfate, and the solvent is evaporated off. A crude oil is obtained, and is purified by chromatography on silica gel (eluent: EtOAc/MeOH). Product (VIa) corresponding to (2S,3S)-4-((dimethoxyphosphoryl)oxy)-2-hydroxy-3-((4-methoxybenzyl)oxy)butyl-4-ethylbenzene sulfonate is obtained in the form of a translucent pale yellow oil in 76% yield (3.95 g, 7.8 mmol).

Formula: C₂₁H₂₉O₁₀PS, Molar mass=504.49 g·mol⁻¹.

¹H NMR (300.13 MHz, CDCl₃), δ (ppm): 2.43 (3H, phenyl-CH₃); 2.92 (1H, OH); 3.70 (m, 1H, CH(OPMB)); 3.72 and 3.76 (s, 3H each, P—OCH₃); 3.79 (s, 3H, benzyl OCH₃); 3.94 (m, 1H, CH((OH)); 3.99 (2H, CH₂(OR)); 4.01-4.15 and 4.17-4.30 (2m, 2*1H, CH₂OP(O)(OCH₃)₂); 4.40-4.62 (2d, J=11.1 Hz, 2H, benzyl CH₂); 6.85 (d, J=8.5 Hz, 2H, aromatic CH); 7.20 (d, J=8.6 Hz, 2H, aromatic CH); 7.32 (d, J=8.1 Hz, 2H, tosyl group aromatic CH); 7.75 (d, J=8.2 Hz, 2H, tosyl group aromatic CH).

³¹P NMR (121.442 MHz, CDCl₃), δ (ppm): 1.48 (P(O)(OMe)₂).

¹³C NMR (75.49 MHz, CDCl₃), δ (ppm): 21.7 (CH₃); 54.5 and 54.6 (2d, J=6.0 Hz, —P(O)(OCH₃)₂); 55.3 (benzyl OCH₃); 65.3 (d, J=6.0 Hz, CH₂—OP(O)(OCH₃)₂); 68.2 (CH(OH)); 69.9 (CH₂(OR)); 72.9 (benzyl CH₂); 75.7 (CH(OPMB)); 113.9, 129.7, 130.1 and 159.5 (6C, benzyl group aromatic C); 128.3, 129.8, 132.6 and 146.1 (6C, tosyl group aromatic C).

HRMS (ESI+) m/z calcd. For [C₂₁H₂₉O₁₀PS][M+NH₄]⁺: 522.1563; found: 522.1559.

IR (v cm⁻¹): 814.36 (v p-disubst. benzene); 843.36 (v S—O); 1026.76 (v P—O—(C)); 1095.72 (v disubst. alcohol); 1110-1140 (v alkyl ethers); 1174 (v C═C—O—C); 1244.85 (v P═O); 1358.05 (v S═O); 1458.70 (v alkyls CH₂); 1514.14-1612.73 (v aryl); 2815-2835 (v O—CH₃); 2957.98 (v alkyls, CH₃); 3010 (v=C—H aryl); 3368.99 (v 0-H).

### Fourth Step:

The fourth step is a step of nucleophilic substitution to replace the tosylate with azide.

5.7 g (11.3 mmol) of tosyl phosphate (VIa) as prepared in the preceding step are dissolved in 28 ml of dry dimethylformamide (DMF) under an inert atmosphere. The resulting mixture is stirred, 2.94 g (45.2 mmol, 4 equiv.) of sodium azide are added and the resulting mixture is stirred for 24 h at 50° C. under an inert atmosphere.

The product obtained is diluted in chloroform (100 ml), and distilled water is then added (60 ml). The dimethyl phosphate (VIIa) is then extracted three times with chloroform (150 ml). The dimethyl phosphate is purified by chromatography on silica gel (eluent: EtOAc/MeOH). A first amount of dimethyl phosphate (VIIa) is obtained.

The aqueous phase obtained during extraction of the dimethyl phosphate with chloroform comprises the monomethyl phosphate (VII′a).

100 ml of chloroform are added to the aqueous phase, which is then cold-acidified with HCl (1M) to a pH equal to 1. The monomethyl phosphate is then extracted from the aqueous phase with chloroform (5*100 ml). The organic phases are combined and dried over MgSO₄, and the solvents are then evaporated off.

The monomethyl phosphate is then remethylated by diluting it in methanol and using TMSCHN₂ (0.6 M or 2 M in hexane) which is added until the mixture remains yellow, and stirred for 30 min. Product (VIIa) obtained requires no purification and represents a second amount of dimethyl phosphate (VIIa).

The first and second amounts of product (VIIa) are combined and correspond to (2S,3S)-4-azido-3-hydroxy-2-((4-methoxybenzyl)oxy)butyl dimethyl phosphate in the form of a translucent pale yellow oil with a total yield of 53%.

Formula: C₁₄H₂₂N₃O₇P, Molar mass=375.31 g·mol⁻¹.

¹H NMR (300.13 MHz, CDCl₃), δ (ppm): 2.67 (1H, OH); 3.25-3.43 (2dd, J=12.6 Hz, 6.5 Hz, 2*1H, CH₂N₃); 3.64 (m, 1H, CH(OPMB)); 3.76 and 3.80 (s, 3H each, POCH₃); 3.81 (s, 3H, benzyl OCH₃); 3.83 (m, 1H, CH(OH)); 4.05-4.19 and 4.20-4.31 (2m, 2*1H, CH₂OP(O)(OCH₃)₂); 4.47-4.72 (2d, J=11.2 Hz, 2H, benzyl CH₂); 6.88 (d, J=8.5 Hz, 2H, aromatic CH); 7.28 (d, J=8.4 Hz, 2H, aromatic CH).

³¹P NMR (121.442 MHz, CDCl₃), δ (ppm): 1.60 (P(O)(OMe)₂).

¹³C NMR (75.49 MHz, CDCl₃), δ (ppm): 53.1 (CH₂N₃); 54.6 and 54.7 (2d, J=6.0 Hz, —P(O)(OCH₃)₂); 55.3 (benzyl OCH₃); 65.3 (d, J=6.0 Hz, (CH₂—OP(O)(OCH₃)₂); 69.9 (CH(OH)); 73.0 (benzyl CH₂); 76.6 (CH(OPMB)); 114.1, 129.5, 130.1 and 159.8 (6C, aromatic C).

HRMS (ESI+) m/z calcd. For [C₁₄H₂₂N₃O₇P] [M+NH₄]⁺: 393.1539; found: 393.1529.

IR (v cm⁻¹): 814.36 (v p-disubst. benzene); 1029.53 (v P—O—(C)); 1177.17 (v alkyl ethers); 1248.07 (v P═O); 1459.48 (v alkyls, CH₂); 1514.14-1612.84 (v aryl); 2100.77 (v azide); 2815-2853 (v O—CH₃); 2957.98 (v alkyls, CH₃); 3010 (v=C—H aryl); 3375.45 (v 0-H).

### Fifth Step:

The fifth step is a step of esterification of the free alcohol function in position sn-1 with a saturated fatty acid.

324 mg (1.14 mmol, 1.5 equiv.) of stearic acid (VIIIa) are dissolved in 20 ml of dry DCM under an inert atmosphere. 236 mg (1.14 mmol, 1.5 equiv.) of N,N′-dicyclohexylcarbodiimide (DCC) and 93 mg (0.78 mmol, 1 equiv.) of 4-dimethylaminopyridine (DMAP) are then added and the mixture is stirred for 5 min at room temperature under an inert atmosphere. Next, a solution comprising 285 mg (0.76 mmol, 1 equiv.) of azide (VIIa) as prepared in the preceding step and 20 ml of dry DCM is added to the mixture. The resulting mixture is stirred for 4 h at room temperature under an inert atmosphere. The temperature is then reduced to 0° C. and 15 ml of ethyl acetate are added. The organic phase is washed three times with saturated NaHCO₃ solution, dried over MgSO₄ and the solvent is evaporated off under reduced pressure. The crude product is purified by chromatography on silica gel (eluent: cyclohexane/ethyl acetate). Product (IXa) corresponding to (2S,3S)-1-azido-4-((dimethoxyphosphoryl)oxy)-3-((4-methoxybenzyl)oxy)butan-2-yl stearate is obtained in the form of a white solid in 50% yield (230 mg, 0.36 mmol).

Formula: C₃₂H₅₆N₃O₈P, Molar mass=641.75 g·mol⁻¹.

¹H NMR (300.13 MHz, CDCl₃), δ (ppm): 0.85 (t, J=6.6 Hz, 3H, CH₃; 1.15-1.65 (m, 28H, CH₂); 1.89 (m, 2H, CH₂(CH₂)C(O)); 2.30 (m, 2H, CH₂C(O)); 3.40 (2dd, J=12.8 Hz; 6.8 Hz, 2*1H, CH₂N₃); 3.71 and 3.75 (d, 3H each, P OCH₃); 3.75 (s, 3H, benzyl OCH₃); 3.77 (m, 1H, CH(OPMB)); 4.08-4.11 (m, 2H, CH₂OP(O)(OCH₃)₂); 4.49-4.67 (2d, J=11.2 Hz, 2H, benzyl CH₂); 5.14 (m, 1H, CH(OC(O)CH₂); 6.86 (d, J=8.8 Hz, 2H, aromatic CH); 7.24 (d, J=8.6 Hz, 2H, aromatic CH).

³¹P NMR (121.442 MHz, CDCl₃), δ (ppm): 1.22 (P(O)(OMe)₂).

¹³C NMR (75.49 MHz, CDCl₃), δ (ppm): 14.17 (CH₃); 22.76-34.02 (15C, CH₂); 34.2 (CH₂C(O)); 50.26 (CH₂N₃); 54.5 and 54.6 (2d, J=6.0 Hz, —P(O)—(OCH₃)₂); 55.3 (benzyl OCH₃); 66.7 (d, J=6.0 Hz, (CH₂OP(O)(OCH₃)₂); 70.9 (CH(OC(O)CH₂); 72.9 (benzyl CH₂); 75.4 (Qi(OPMB)); 114.0, 129.4, 129.8 and 159.6 (6C, aromatic C); 173.0 (OC(O)CH₂).

HRMS (ESI+) m/z calcd. For [C₃₂H₅₆N₃O₈P] [M+Na]⁺: 664.3703; found: 664.3702.

IR (v cm⁻¹): 850.03 (v p-disubst. benzene); 1039.63 (v P—O—(C)); 1157.17 (v C—O); 1276.71 (v P═O); 1459.48 (v alkyls, CH₂); 1514.14-1612.84 (v C═C aryl); 1745.17 (v C═O); 2101.25 (v azide); 2853.16 (v O—CH₃); 2923.78 (v alkyls, CH₃).

### Sixth Step:

The sixth step is a step of deprotection of the para-methoxybenzyl (PMB) group followed by a step of esterification of the free alcohol function in the sn-2 position with an unsaturated fatty acid.

185 mg (0.290 mmol) of stearic azide (IXa) as prepared in the preceding step are dissolved in 6 ml of dry DCM under an inert atmosphere. Next, 338 μl of aqueous phosphate-buffered saline (PBS) (pH 7.2) containing 0.15 M NaCl are added. 325.95 mg (1.45 mmol, 5 equiv.) of 2,3-dichloro-5,6-dicyano-1,4-benzoquinone (DDQ) are added at 0° C. under an inert atmosphere over 5 h. The reaction medium is then diluted with 6 ml of cold DCM and 5 ml of cold saturated NaHCO₃ solution. The resulting mixture turns dark green. The aqueous phase is extracted twice with 20 ml of cold DCM, and the organic phases are combined and washed with 20 ml of saturated NaHCO₃ solution, dried over MgSO₄ and the solvents are evaporated off under reduced pressure. The alcohol obtained (Xa) is in the form of an orange-yellow oil.

162 mg (0.65 mmol, 1.5 equiv.) of oleic acid (XIa) are dissolved in 7 ml of dry DCM under an inert atmosphere at 0° C. Next, 89 mg (0.44 mmol, 1.5 equiv.) of DCC and 36 mg (0.29 mmol, 1 equiv.) of DMAP are added and the resulting mixture is stirred for 5 min at room temperature under an inert atmosphere. A solution comprising 152 mg (0.290 mmol, 1 equiv.) of the alcohol (Xa) obtained in the preceding step and 7 ml of dry DCM under an inert atmosphere is then added at 0° C. to said mixture. The resulting mixture is stirred for 5 hours at 0° C. under an inert atmosphere. 10 ml ethyl acetate are then added. The organic phase is washed three times with saturated NaHCO₃ solution, dried over MgSO₄ and the solvent is evaporated off under reduced pressure. The crude product is purified by chromatography on silica gel (eluent: cyclohexane/ethyl acetate). Product (XIIa) corresponding to (2S,3S)-4-azido-1-((dimethoxyphosphoryl)oxy)-3-(stearoyloxy)butan-2-yl oleate is obtained in the form of a translucent pale-white oil in 50% yield (111 mg, 0.15 mmol).

Formula: C₄₂H₈₀N₃O₈P, Molar mass=786.07 g·mol⁻¹.

¹H NMR (300.13 MHz, CDCl₃), δ (ppm): 0.86 (t, J=6.6 Hz, 6H, 2*CH₃); 1.25 (m, 48H, CH₂); 1.61 (m, 2*2H, CH₂CH₂C(O)O); 2.00 (m, 4H, CH₂CH═CHCH₂); 2.32-2.38 (m, 2*2H, CH₂C(O)O); 3.39-3.45 (dd, J=13.2 Hz; 5.4 Hz, 1H, CH₂N₃); 3.47-3.53 (dd, J=13.2 Hz; 3.6 Hz, 1H, CH₂N₃); 3.73 and 3.78 (d, 3H each, P—OCH₃); 4.15-4.18 (m, 2H, CH₂OP(O)(CH₃)₂); 5.27 (m, 2*1H, 2*CH(OC(O)CH₂); 5.32 (m, 2H, CH═CH).

³¹P NMR (121.442 MHz, CDCl₃), δ (ppm): 1.10 (P(O)(OMe)₂).

¹³C NMR (75.49 MHz, CDCl₃), δ (ppm): 14.17 (2*CH₃); 22.70 (2*CH₂CH₃); 24.82 (2*CH₂CH₂C(O)O); 27.34 (CH₂CH═CHCH₂); 29.12-31.94 (22C, CH₂); 34.08 (2*CH₂C(O)); 50.64 (CH₂N₃); 54.54 and 54.62 (2d, J=6.0 Hz, —P(O)—(OCH₃)₂); 65.10 (d, J=6.0 Hz, CH₂OP(O)(OCH₃)₂); 69.69 (stearic CH(OC(O)CH₂); 70.10 (oleic CH(OC(O)CH₂); 130.06, 129.67 (CH₂CH CHCH₂); 172.7 (2*OC(O)CH₂).

HRMS (ESI+) m/z calcd. For [C₄₂H₈₀N₃O₈P] [M+H]⁺: 786.5761; found: 786.5759.

IR (v cm⁻¹): 722.15 (v Z CH═CH); 1039.63 (v P—O—(C)); 1157.46 (v C—O); 1276.71 (v P═O); 1464.53 (v alkyls, C); 1745.17 (v C═O); 2101.75 (v azide); 2853.31 (v O—CH₃); 2922.63 (v alkyls, CH₃)

### Seventh Step:

The seventh step is a step of deprotection of the phosphate head.

50 mg (0.64 μmol) of phosphate diester (XIIa) as prepared in the preceding step are dissolved in 3 ml of CDCl₃ previously neutralized with K₂CO₃ and the resulting solution is stored over molecular sieves under an inert atmosphere. 21 μl (160 μmol, 2.5 equiv.) of bromotrimethylsilane (TMSBr) are then added and the resulting mixture is stirred for 5 h at room temperature under a neutral atmosphere. 1 ml of water (H₂O) is added and the solvents are evaporated off. The crude product is purified by chromatography on silica gel (water/methanol, column C18AQ sold by Interchim). Product (Ia) corresponding to (2S,3S)-4-azido-2-(oleoyloxy)-3-(stearoyloxy)butyl hydrogen phosphate (modified PA 18:1-18:0) is obtained in the form of a pale white oil in 31% yield (15 mg, 0.198 μmol).

Formula: C₄₀H₇₅N₃O₈P⁻, Molar mass=757.07 g·mol⁻¹.

### Example 2: Synthesis of GPL (Ib)

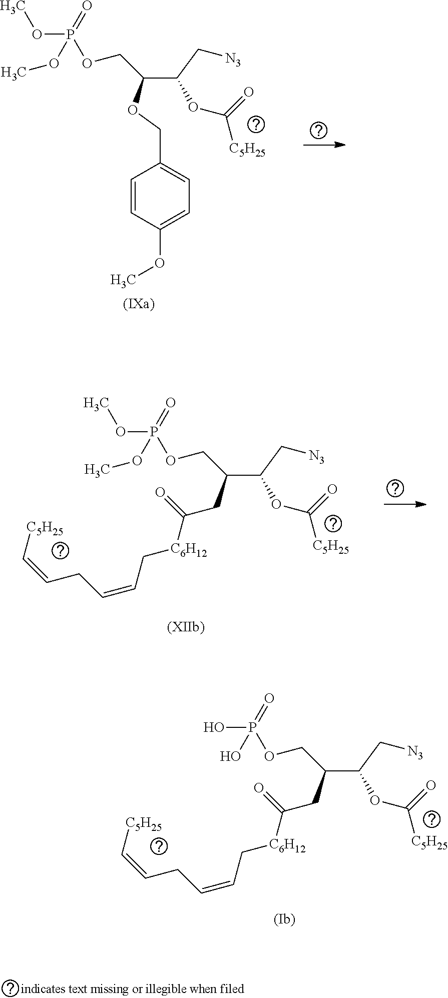

330 mg (0.51 mmol) of stearic azide as prepared in Example 1 are dissolved in 10 ml of dry DCM under an inert atmosphere. Next, 550 μl of aqueous phosphate-buffered saline (PBS) (pH 7.2) comprising 0.15 M NaCl are added. 530 mg (2.55 mmol, 5 equiv.) of DDQ are added at 0° C. under an inert atmosphere over 6 h. The reaction medium is then diluted with 10 ml of cold DCM and 10 ml of cold saturated NaHCO₃ solution. The resulting mixture turns dark green. The aqueous phase is extracted twice with 40 ml of cold DCM, and the organic phases are combined and washed with 40 ml of cold saturated NaHCO₃ solution, dried over MgSO₄ and the solvents are evaporated off under reduced pressure. The alcohol obtained is in the form of an orange-yellow oil. 283 mg (1.02 mmol, 25 equiv.) of linoleic acid are dissolved in 5 ml of dry DCM under an inert atmosphere at 0° C. Next, 159 mg (0.78 mmol, 1.5 equiv.) of DCC and 63 mg (0.52 mmol, 1 equiv.) of DMAP are added and the resulting mixture is stirred for 5 min at room temperature under an inert atmosphere. A solution comprising 266 mg (0.52 mmol, 1 equiv.) of the alcohol obtained in the preceding step and 5 ml of dry DCM under an inert atmosphere is then added at 0° C. to said mixture. The resulting mixture is stirred for 5 hours at 0° C. under an inert atmosphere. 15 ml ethyl acetate are then added. The organic phase is washed three times with saturated NaHCO₃ solution, dried over MgSO₄ and the solvent is evaporated off under reduced pressure. The crude product is purified by chromatography on silica gel (eluent: cyclohexane/ethyl acetate). The product corresponding to (2S,3S)-4-azido-1-((dimethoxyphosphoryl)oxy)-3-(stearoyloxy)butan-2-yl oleate is obtained in the form of a translucent pale yellow oil in 50% yield (200 mg, 0.26 mmol).

Formula: C₄₂H₇₈N₃O₈P, Molar mass=784.07 g·mol⁻¹.

¹H NMR (300.1-3 MHz, CDCl₃), δ (ppm): 0.92 (t, J=6.6 Hz, 6H, 2*CH₃); 1.30 (m, 44H, CH₂); 1.67 (m, 2*2H, CH₂CH₂C(O)O); 2.08 (m, 4H, CH₂CH═CHCH₂); 2.35-2.40 (m, 2*2H, CH₂C(O)O); 3.43-3.48 (dd, J=13.2 Hz; 5.4 Hz, 1H, CH₂N₃); 3.53-3.57 (dd, J=13.2 Hz; 3.6 Hz, 1H, CH₂N₃); 3.72 and 3.78 (d, 3H each, POCH₃); 4.12-4.23 (m, 2H, CH₂OP(O)(CH₃)₂); 5.31 (m, 2*1H, 2*CH(OC(O)CH₂); 5.37-5.40 (m, 4H, CH═CH).

³¹P NMR (121.442 MHz, CDCl₃), δ (ppm): 1.08 (P(O)(OMe)₂).

¹³C NMR (75.49 MHz, CDCl₃), δ (ppm): 14.07 and 14.12 (2*CH₃); 22.58 and 22.70 (2*CH₂CH₃); 24.82 and 24.84 (2*CH₂CH₂C(O)O); 27.21 (CH₂CH═CHCH₂); 29.12-31.94 (22C, CH₂); 34.07 (2*CH₂C(O)); 50.64 (CH₂N₃); 54.55 and 54.63 (2d, J=6.0 Hz, —P(O)—(OCH₃)₂); 65.16 (d, J=6.0 Hz, CH₂OP(O)(OCH₃)₂); 69.69 (CH(stearic OC(O)CH₂); 70.01 (linoleic CH(OC(O)CH₂); 127.89, 128.12, 129.96 and 130.23 (CH₂CH═CHCH₂); 172.68 and 172.70 (2*OC(O)CH₂).

HRMS (ESI+) m/z calcd. For [C₄₂H₇₈N₃O₈P] [M+Na]⁺: 806.5424; found: 806.5426.

50 mg (0.64 μmol) of phosphate diester (XIIb) as prepared in the preceding step are dissolved in 3 ml of CDCl₃ previously neutralized with K₂CO₃ and the resulting solution is stored over molecular sieves under an inert atmosphere. 21 μl (160 μmol, 2.5 equiv.) of bromotrimethylsilane (TMSBr) are then added and the resulting mixture is stirred for 5 h at room temperature under a neutral atmosphere. 1 ml of water (H₂O) is added and the solvents are evaporated off. The crude product is purified by chromatography on silica gel (water/methanol, column C18AQ sold by Interchim). Product (Ib) corresponding to (2S,3S)-4-azido-2-(linoleoyloxy)-3-(stearoyloxy)butyl hydrogen phosphate (modified PA 18:2-18:0) is obtained in the form of a pale white oil in 31% yield (15 mg, 0.198 μmol).

Formula: C₄₀H₇₃N₃O₈P⁻, Molar mass=755.07 g·mol⁻¹

### Example 3: Synthesis of a GPL Fluorescent Probe (XIVa) from GPL (Ia)

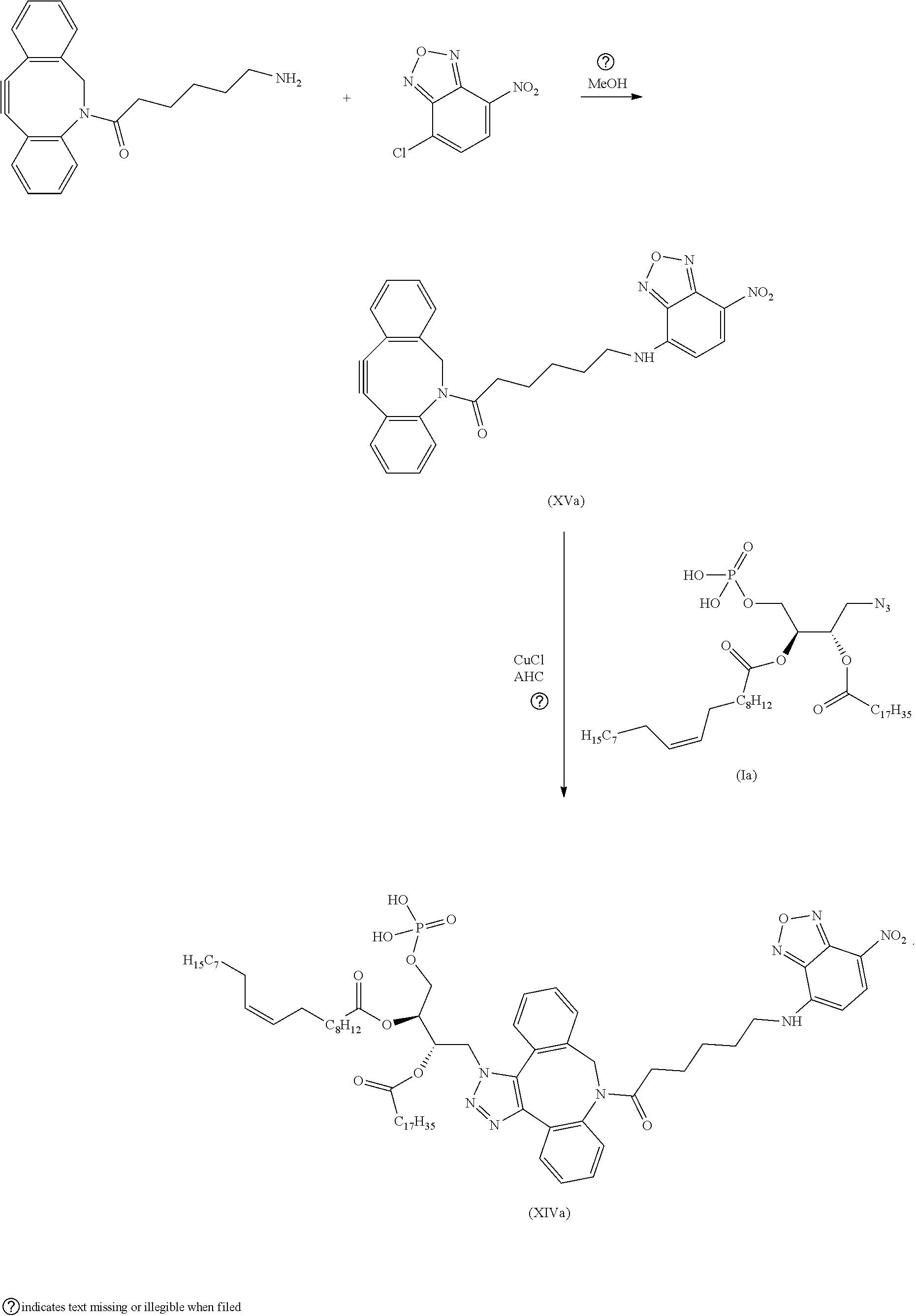

33.09 mg (0.1663 mmol) of 4-chloro-7-nitrobenzo[c][1,2,5]oxadiazole (NBDCl) are added to a solution of 132 mg (0.415 mmol, 2.5 equiv.) of cyclooctyne (ADIBO C6 amine) in 5 ml of MeOH. 140 μl (0.832 mmol, 5 equiv.) of DIPEA are added and the resulting mixture is stirred for 2 h at room temperature. The solvent is subsequently evaporated off under reduced pressure. The crude product is purified by chromatography on silica gel (eluent: CH₂Cl₂/MeOH). Product (XVa) corresponding to 1-(11,12-didehydrodibenzo[b,f]azocin-5(6H)-yl)-6-((7-nitrobenzo[c][1,2,5]oxadiazol-4-yl)amino)hexan-1-one (ADIBO-NBD) is obtained in the form of a fluorescent yellow-green oil in 57% yield (45 mg, 94 μmol).

Formula: C₂₇H₂₃N₅O₄, Molar mass=481.18 g·mol⁻¹.

¹H NMR (300.13 MHz, CDCl₃), δ (ppm): 1.21-1.23 (m, 2H); 1.25-1.42 (m, 4H); 1.94-2.01 (m, 2H); 2.21-2.30 (m, 2H); 3.65-3.70 (d, J=13.8 Hz, 1H); 5.17-5.22 (d, J=13.8 Hz, 1H); 6.04-6.07 (d, J=8.7 Hz, 1H); 7.26-7.42 (m, 4H); 7.70-7.72 (dd, J=7.2 Hz; 1.3 Hz, 1H); 8.40-8.50 (dd, J=8.7 Hz: 3.5 Hz. 1H).

10 mg (0.0132 mmol) of ADIBO-NBD (XVa) as prepared in the preceding step in 1 ml CDCl₃ are added to a solution comprising 25.4 mg (0.0527 mmol, 4 equiv.) of GPL (Ia) as prepared in Example 1 and 3 ml of CDCl₃. The resulting mixture is stirred for 1 h at 40° C. The solvent is evaporated off under reduced pressure. The crude product is purified by chromatography on silica gel (eluent: H₂O/MeCN). Product (XIVa) corresponding to (2S,3S)-4-(8-(6-((7-nitrobenzo[c][1,2,5]oxadiazol-4-yl)amino)hexanoyl)-8,9-dihydro-1H-dibenzo[b,f][1,2,3]triazolo[4,5-d]azocin-1-yl)-1-(phosphonooxy)-3-(stearoyloxy)butan-2-yl oleate is obtained in the form of a fluorescent yellow-green oil in 90% yield (14.7 mg, 12 μmol).

Formula: C₆₇H₉₉N₈O₁₂P, Molar mass=1238.71 g·mol⁻¹.

HRMS (ESI−) m/z calcd. For [C₆₇H₉₈N₈O₁₂P] [M−H]⁻: 1237.7047; found: 1237.7104.

### Example 4: Use of GLP (Ia) and (Ib) as Molecular Tools

This example illustrates the interaction between GPL (Ia) or (Ib) and a phosphatidic acid (PA) mobilizing protein, the green-fluorescent protein Spo20p-GFP, expressed in target cells.

For this purpose, GPL (Ia) and, for comparative purposes, a natural GPL PA 18:1-18:0 (referred to hereinbelow as GPL C1) sold under reference 840861C by Avanti Polar Lipids, are incubated for 5 min at a concentration of 100 μM, with PC12 cells expressing Spo20p-GFP.

The GPL-free cell medium is used as a control (referred to hereinbelow as C1). Under these control conditions, Spo20p-GFP is essentially found in the nucleus and cytoplasm of the cells.

Similarly, GPL (Ib) and, for comparative purposes, a natural GPL PA 18:2-18:0 (referred to hereinbelow as GPL C2) sold under reference 840862C by Avanti Polar Lipids, are incubated for 5 min at a concentration of 100 μM, with PC12 cells expressing Spo20p-GFP.

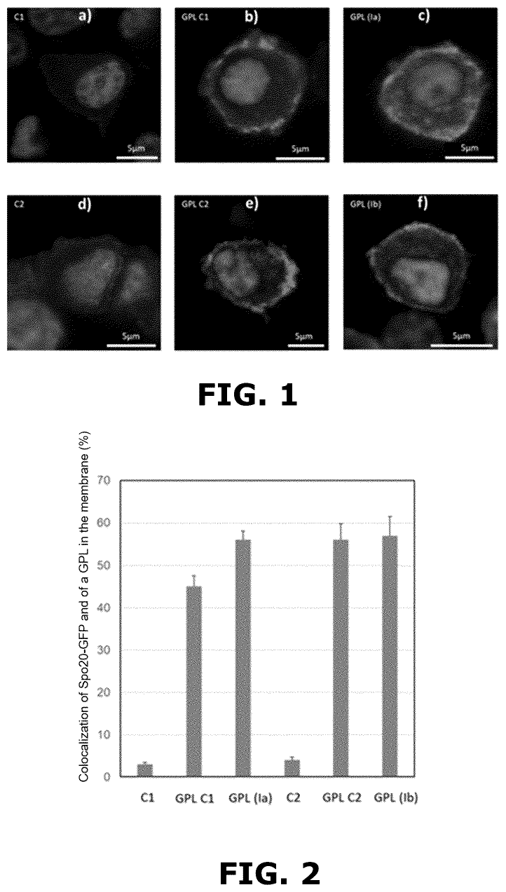

The GPL-free cell medium is used as a control (referred to hereinbelow as C2). FIG. 1 shows confocal microscopy images obtained using a machine sold under the trade name SP5II by the company Leica, of the two control media C1 and C2 (FIGS. 1a and 1d, respectively), the medium after incubation with the comparative GPL C1 (FIG. 1b), the medium after incubation with GPL (Ia) of the invention (FIG. 1c), the medium after incubation with the comparative GPL C2 (FIG. 1e), and the medium after incubation with GPL (Ib) of the invention (FIG. 1f).

As can be seen from FIG. 1, the GPLs (Ia) and (Ib) of the invention bring about recruitment of the Spo20p-GFP protein to the plasma membrane of PC12 cells in the same manner as do the corresponding natural GPLs, GPL C1 and GPL C2. This indicates that the GPLs of the invention reproduce two important features of natural PAs, namely insertion into the plasma membrane by accessing the inner leaflet and recruitment of the Spo20p-GFP protein therein.

A marker referred to hereinbelow as Membright (obtained from Mayeul Collot UMR-7021, Strasbourg) is incubated for 5 minutes on cells in media as prepared above to specifically mark the cells' plasma membrane.

FIG. 2 shows the percentage of colocalization of the Spo20p-GFP protein and various GPLs in the plasma membrane, said percentage being determined using Image J software.

Consequently, by means of the synthetic GPLs (I) of the invention, it is possible to localize and identify the various proteins that interact therewith.

### Example 5: Use of GPL (XIVa) as a Molecular Tool

This example illustrates the interaction between GPL (XIVa), which fluoresces green (by means of a nitrobenzadiazole-based fluorophore), and a red-fluorescent phosphatidic acid (PA) mobilizing protein, CgA-AF633.

For this purpose, giant liposomes composed of 96% by mass of dioleoylphosphatidylcholine (DOPC) and 4% by mass of GPL (XIVa), and, for comparative purposes, giant liposomes composed of 96% by mass of dioleoylphosphatidylcholine (DOPC) and 4% by mass of a natural GPL modified with a nitrobenzadiazole-based fluorophore: PA 18:1-6:0 NBD sold under the reference 810175C by Avanti Polar Lipids, at a concentration of 1 mM, are prepared using the polyvinyl alcohol (PVA)-assisted swelling method as described in Carmon et al., FASEB J., 2020, 34:6769-6790).

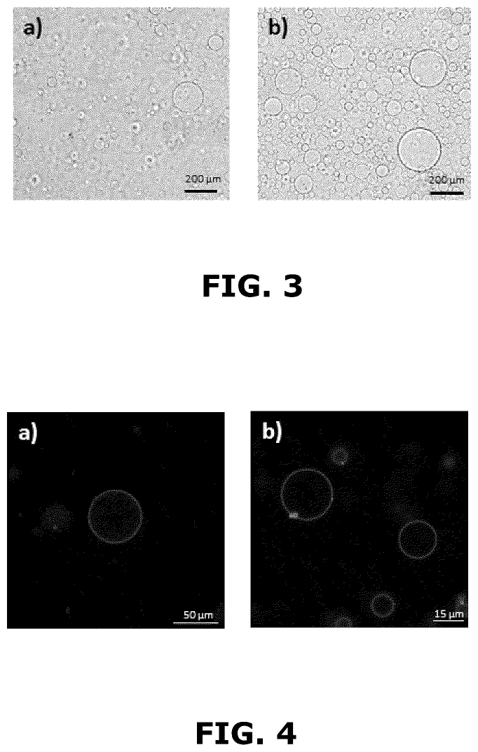

FIG. 3 shows light microscopy images of liposomes containing the commercial GPL PA 18:1-6:0 NBD (FIG. 3a) and liposomes containing GPL (XIVa) of the invention (FIG. 3b).

FIG. 4 shows images obtained using a confocal microscope sold under the trade name SP5 by the company Leica, of a liposome containing the commercial GPL PA 18:1-6:0 NBD (FIG. 4a) and liposomes containing GPL (XIVa) of the invention (FIG. 4b), thus indicating that GPL (XIVa) of the invention participates in the formation of giant liposomes.

CgA, rendered red-fluorescent due to its coupling with the fluorophore Alexa 633 or AF633 (CgA-AF633), is incubated at 4 μM concentration with liposomes as prepared above.

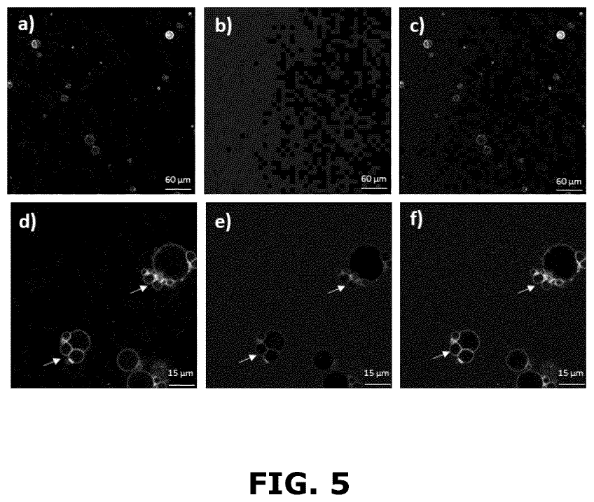

FIG. 5 shows images obtained using the confocal microscope of liposomes containing the commercial GPL PA 18:1-6:0 NBD incubated with the CgA-AF633 protein (FIGS. 5a, 5b, 5c) and liposomes containing GPL (XIVa) of the invention incubated with the CgA-AF633 protein (FIGS. 5d, 5e, 5f).

In FIGS. 5a and 5d, the excitation wavelength is 463 nm and the detection wavelength is 539 nm to allow visualization of the green-fluorescent GPLs; in FIGS. 5b and 5e, the excitation wavelength is 633 nm and the detection wavelength is 648 nm to allow visualization of the red-fluorescent CgA-AF633 protein; FIGS. 5c and 5f result from the overlapping of FIGS. 5a and 5b and then FIGS. 5d and 5e, respectively, so as to visualize both the green-fluorescent GPLs and the red-fluorescent CgA-AF633 protein.

Whereas incubation results in no interaction of the CgA-AF633 protein with liposomes containing the commercial GPL PA 18:1-6:0 NBD, it does result in membrane deformations in the liposomes containing GPL (XIVa) of the invention (white arrows).

Moreover, the protein CgA-AF633 only interacts with liposomes containing GPL (XIVa). The interaction of CgA-AF633 with liposomes is materialized by yellow fluorescence, notably in membrane deformations (FIG. 5f).

This indicates that the GPL of the invention reproduces two important features of natural PA, namely interaction with the CgA protein and allowing membrane deformation. Consequently, by means of the synthetic GPL (XIVa) of the invention, it is possible to localize and identify proteins such as CgA that interact therewith.

### Example 6: Synthesis of a GPL Fluorescent Probe (XIVa′) from GPL (Ia)

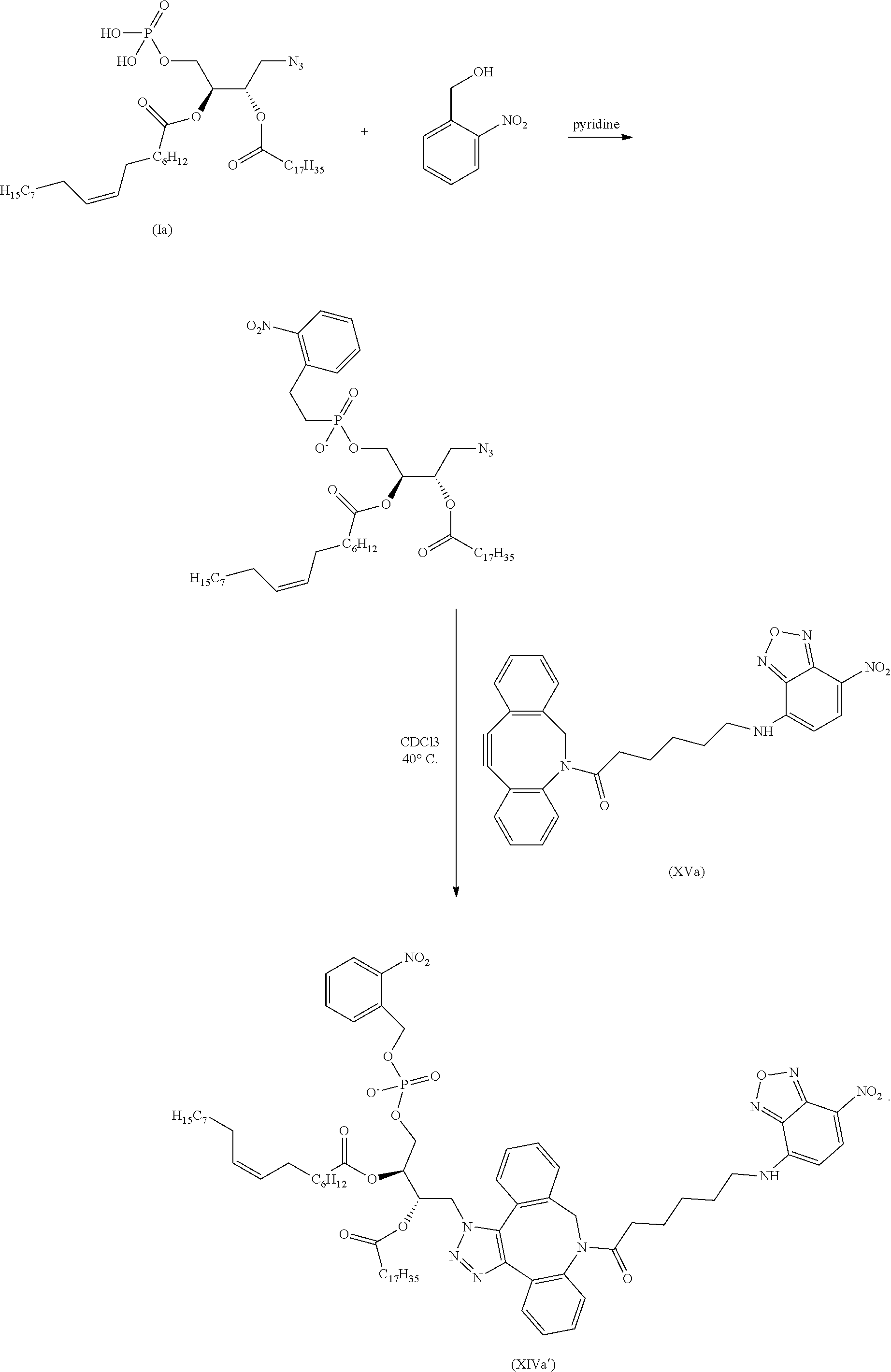

50 mg (66 μmol) of compound (Ia) as prepared in Example 1 were dissolved in 330 μl of freshly distilled pyridine. 50.5 mg (330 μmol, 5 equivalents) of o-nitrobenzyl alcohol and 330 μl (3.29 mmol, 50 equivalents) of trichloroacetonitrile were added. The reaction vessel was sealed with a Teflon septum and irradiated for 60 min at 90° C. (120 W maximum power). The solvent was then evaporated off under vacuum and the crude product was purified by chromatography on silica gel (eluent: CHCl₃/isopropanol) to give an intermediate compound in the form of an oil in 31% yield (18.3 mg).

HRMS (ESI−) m/z calcd. For [C₄₇H₈₁N₄O₁₀P] [M−H]⁻: 891.5609; found: 891.5612.

To a solution of the intermediate compound prepared in the preceding step (5 mg, 5.60 μmol) in 0.5 μl of CDCl₃ were added 10.8 mg (22.4 μmol, 4 equivalents) of ADIBO-NBD as prepared in Example 3 (compound XVa) in 0.5 ml of CDCl₃, and the mixture was stirred for 4 h at 40° C. The solvent was removed under reduced pressure and the crude product was purified by chromatography on silica gel (eluent: CDCl₃/i-PrOH). Product (XIVa′) was obtained in the form of a yellow-green fluorescent oil in a yield of 32% (2.2 mg).

Relative to compound (XIVa), this compound comprises a photoactivatable group OR′¹ as defined in the invention on the polar head.

HRMS (ESI−) m/z calcd. For [C₇₄H₁₀₃N₉O₁₄P] [M−H]⁻: 1372.7368; found: 1372.7371.

### Example 7: Synthesis of a GPL Fluorescent Probe (XIVa-2) from GPL (Ia)

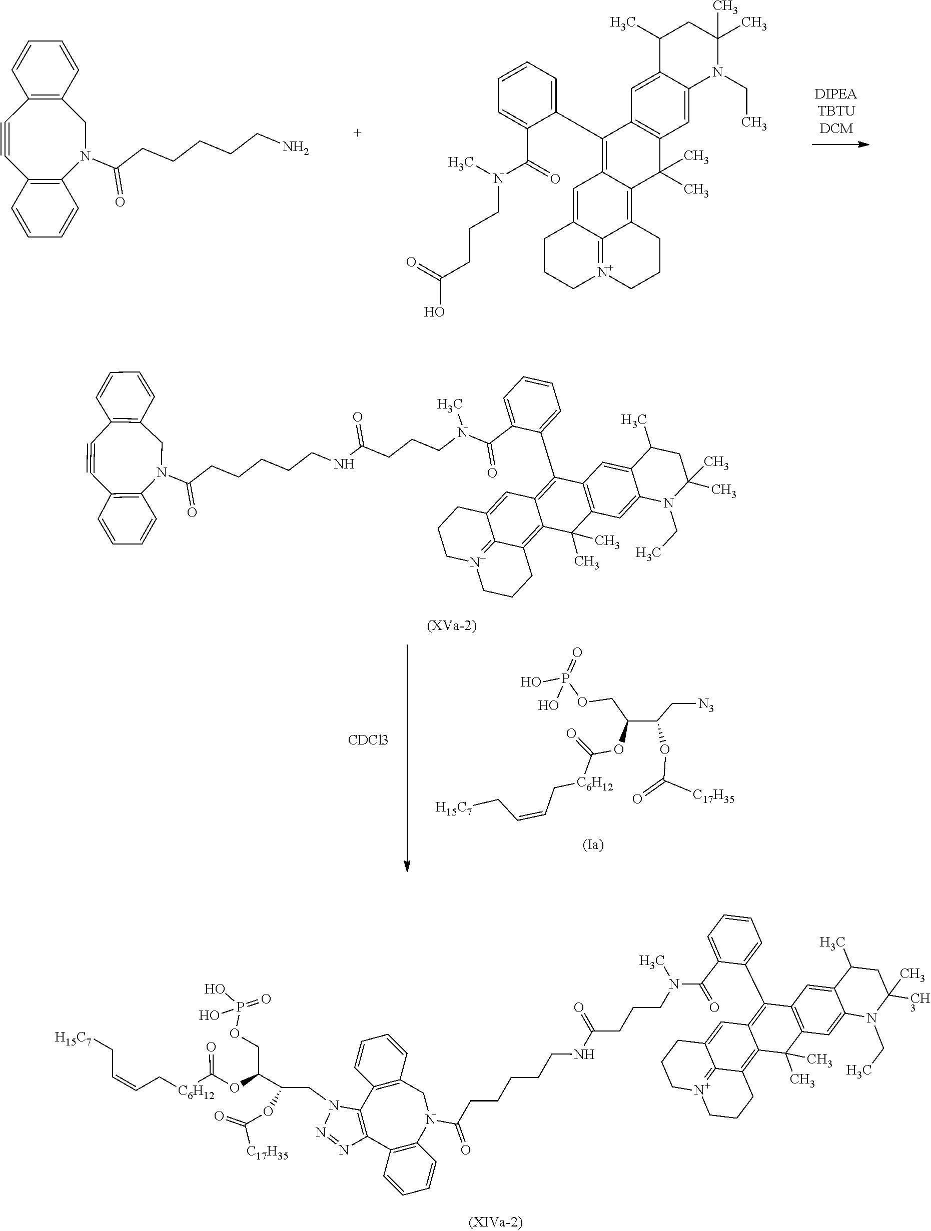

The carboxylic acid “ATTO 746N” (5.00 mg, 6.70 μmol) was dissolved in dichloromethane (150 μl). N-(3-Dimethylaminopropyl)-N′-ethylcarbodiimide (2.50 mg, 16.1 μmol) and the amine ADIBO C6 (4.70 mg, 14.7 μmol) were then added and the mixture was stirred at room temperature for 2 hours. 2-(1H-Benzotriazol-1-yl)-1,1,3,3-tetramethylaminium tetrafluoroborate or TBTU (3.23 mg, 10.1 μmol) and N,N-diisopropylethylamine (DIPEA, 1.75 μl, 10.1 μmol) were then added. After 1 h, the reaction was complete. The solvent was removed under reduced pressure. This crude product of formula (XVa-2) was employed in the next step without purification.

The crude product (XVa-2) (6.33 mg, 6.7 μmol) and GPL (Ia) as prepared in Example 1 (7.99 mg, 10.6 μmol) were dissolved in CHCl₃. After 1 day, the solvent was removed under reduced pressure. The crude product was purified by chromatography on silica gel (eluent: gradient from 5% methanol up to 20% in CH₂Cl₂, “Viridis Silica 5μ, 150×4.60 mm column) to give a blue solid (XIVa-2) (5 mg, 38% yield after 2 steps).

HRMS (ESI+) m/z calcd. For [C₁₀₃H₁₄₈N₈O₁₁P] [M+H]⁺: 1704.1000; found: 1704.1035.

### Example 8: Synthesis of a GPL Fluorescent Probe (XIVa-3) from GPL (Ia)

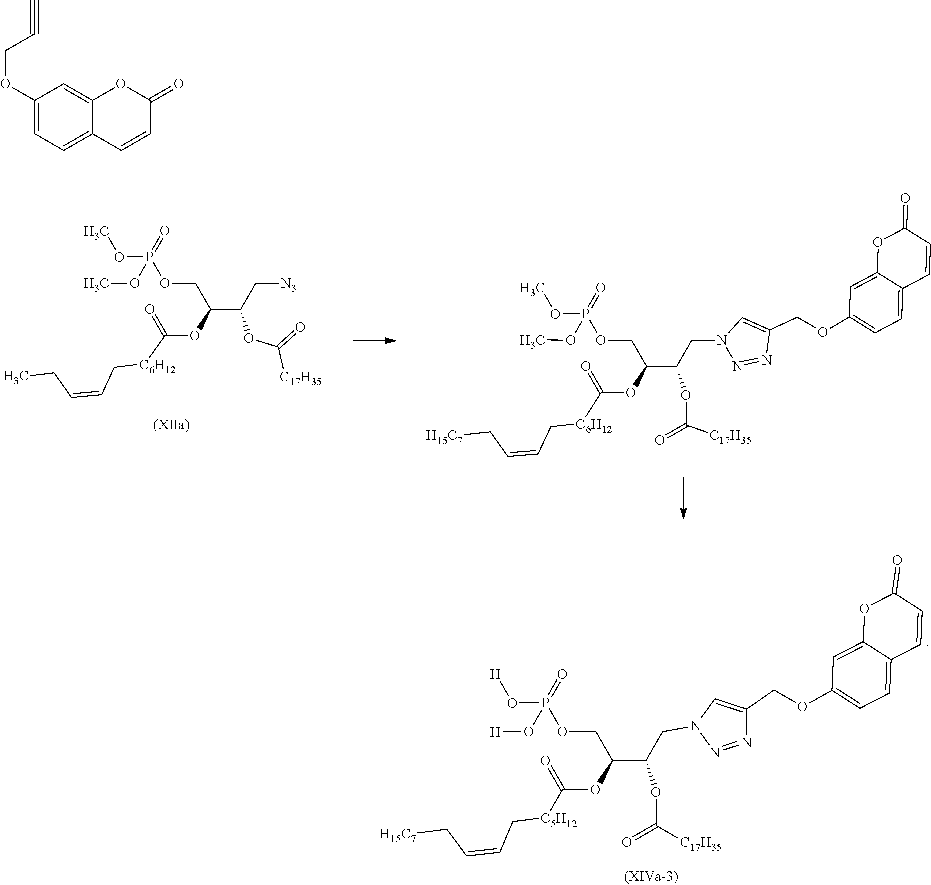

Compound (XIIa) as prepared in Example 1 (83 mg, 106 mmol, 1 equivalent) and 7-(prop-2-yn-1-yloxy)-2H-chromen-2-one (52.8 mg, 264 μmol, 2.5 equivalents) were dissolved in tetrahydrofuran (THF) (4 ml). Copper sulfate pentahydrate (59.0 mg, 370 μmol, 3.5 equivalents) and sodium ascorbate (151 mg, 760 μmol, 7.2 equivalents) were added with water (1 ml). The solution was stirred at room temperature overnight. The solvents were removed under reduced pressure. The crude product was purified by chromatography on silica gel with solid deposit (Celite) and an eluent: gradient of 60% ethyl acetate in cyclohexane) to obtain the intermediate phosphonate diester compound in the form of a brown solid in 66% yield (104 mg).

Molar mass=970.5927 g·mol⁻¹.

¹H NMR (300.13 MHz, CDCl₃), δ (ppm): 7.71 (s, 1H, triazole CH), 7.62 (d, 1H, J=9.5 Hz, coum. CH), 7.37 (m, 1H, coum. CH), 6.90 (m, 2H, coum. CH), 6.27 (d, 1H, J=9.5 Hz, coum. CH), 5.56 (m, 1H, J=5.3 Hz, glycerol backbone CH), 5.33 (m, 2H, double bond CH), 5.24 (s, 2H, coum. benz. CH₂), 5.17 (m, 1H, glycerol backbone CH), 4.61 (d, 2H, J=5.8 Hz, triazole benz. CH₂), 4.21 (m, 2H, CH₂OP), 3.78 (d, 3H, J=2.9 Hz, POCH₃), 3.74 (d, 3H, J=2.8 Hz, POCH₃), 2.36 (m, 2H*2, CH₂CO₂), 2.01 (d, 2H*2, J=6.1 Hz, CH₂CH═CHCH₂), 1.57 (dt, 2H*2, J=33.7 Hz/7.1 Hz, CH₂CH₂CO₂), 1.24 (s, 48H, fatty chain CH₂), 0.87 (t, 6H, J=7 Hz, CH₃*2).

³¹P NMR (121.442 MHz, CDCl₃), δ (ppm): 1.08 (P(O)(OMe)₂).

¹³C NMR (75.49 MHz, CDCl₃), δ (ppm): 172.8 (ester CO), 172.5 (ester CO), 161.4 (ether C_{q} O), 161.2 (coum. ester CO), 155.9 (coum. ester C_{q} alpha O), 143.5 (triazole C_{q}), 143.0 (Cₐᵣ), 130.2 (double bond), 129.8 (double bond), 129.0 (Cₐᵣ), 123.9 (triazole CH), 113.6 (Cₐᵣ), 113.2 (Cqₐᵣ), 112.8 (Cₐᵣ), 102.3 (Cₐᵣ), 69.8 (glycerol alpha ester CH next to the phosphorus, J=7.6 Hz), 69.2, (glycerol alpha ester CH remote from the phosphorus), 64.9 (CH₂ alpha OP, J=5.2 Hz), 62.3 (coum. benz. CH₂), 54.8 (2 CH₃OP, J=6.6 Hz), 50.3 (triazole alpha CH₂), 34.1 (ester alpha CH₂), 34.0 (ester alpha CH₂), 32.0-22.8 (fatty chain CH₂), 14.2 (CH₃).

HRMS (ESI−) m/z calcd. Found: 970.5934.

The intermediate compound as obtained above (90 mg, 91.1 μmol, 1 equivalent) was dissolved in CDCl₃ (5 ml) previously neutralized with K₂CO₃ and stored over molecular sieves under a neutral atmosphere. Freshly distilled TMSBr (69.7 mg, 455 μmol, 5 equivalents) was then added and the mixture was stirred for 5 h at room temperature under a neutral atmosphere. After monitoring by ³¹P NMR, the reaction mixture was cooled with MeOH and evaporated under reduced pressure to obtain a crude product in the form of a brown oil (XVa-3) (95 mg, quantitative yield).

¹H NMR (300.13 MHz, CDCl₃), δ (ppm): 8.60 (s, 1H, triazole CH), 7.65 (s, 1H, coum. CH), 7.37 (s, 1H, coum. CH), 6.92 (s, 2H, coum.), 6.21 (s, 1H, coum.), 5.68 (s, 1H, glycerol backbone CH), 5.54-5.17 (m, 2*1H+2H+2H, CH═CH+coum. benz. CH₂+triazole benz. CH₂), 4.96 (m, 1H, glycerol backbone CH), 4.25 (d, 2H, J=31.7 Hz, CH₂OP), 2.30 (m, 2*2H, CH₂CH₂CO₂), 1.98 (m, 2*2H, CH₂CH═CHCH₂), 1.49 (d, 2*2H, J=27.9 Hz, CH₂CH₂CO₂), 1.24 (s, 48H, fatty chain CH₂), 0.87 (t, 6H, J=6.5 Hz, CH₃).

³¹P NMR (121.442 MHz, CDCl₃), δ (ppm): −0.94 ppm.

### Example 9: Synthesis of a GPL Fluorescent Probe (XIVa-4) from GPL (Ia)

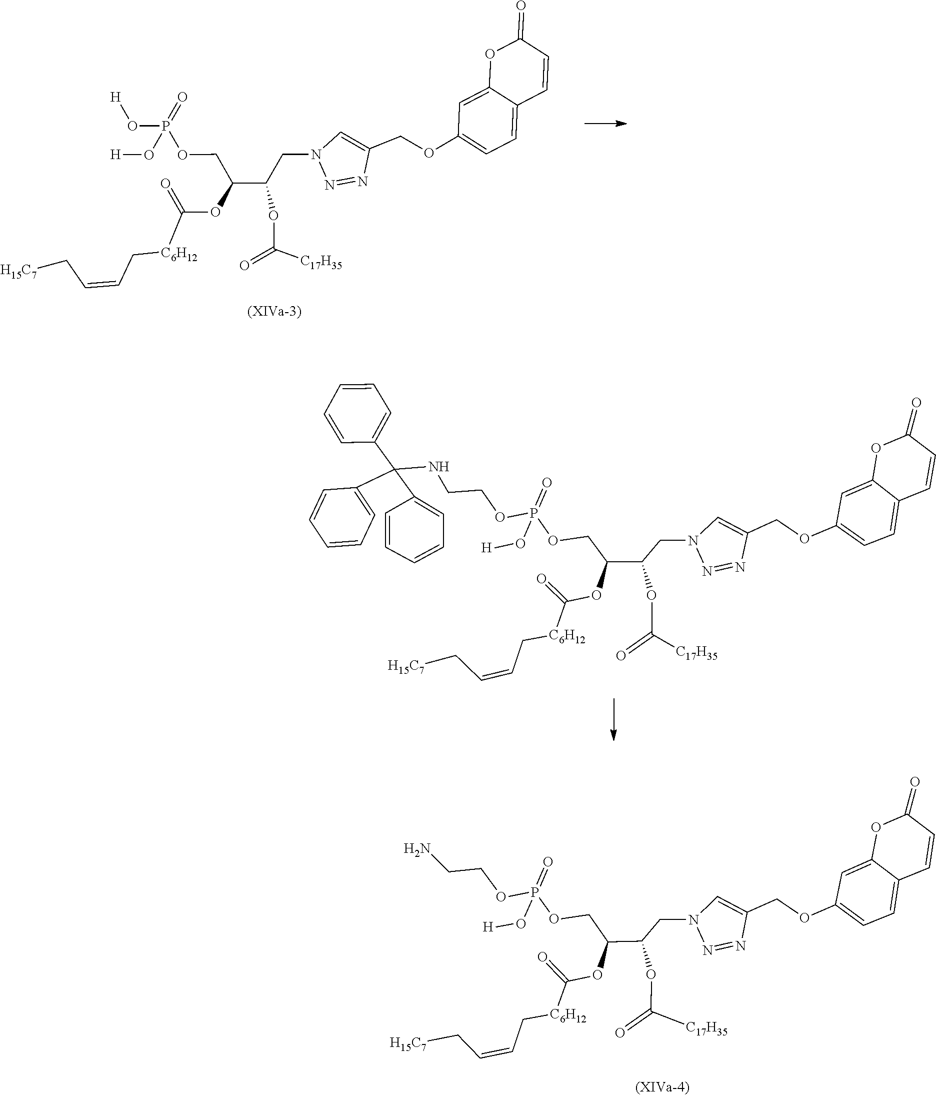

The compound of formula (XIVa-3) as prepared in Example 8 (95 mg, 99.5 μmol, 1 equivalent) was dissolved in pyridine (450 μl), and N-tritylethanolamine (151 mg, 497 μmol, 5 equivalents) and trichloroacetonitrile (450 μl) were then added. The reaction mixture was heated at 90° C. for 1 hour by microwave irradiation. The crude product obtained was purified by chromatography on silica gel with liquid deposit (dichloromethane) and an eluent (gradient from 100% ethyl acetate to 100% isopropanol) to obtain the intermediate compound in the form of a brown solid in 92% yield (114 mg).

Molar mass=1241.7288 g/mol

¹H NMR (300.13 MHz, CDCl₃), δ (ppm): 7.78 (s, 1H, triazole CH), 7.51 (m, 8H, 2 coum. CH+trityl CH), 7.26 (m, 9H, trityl CH), 6.82 (m, 2H, coum. CH), 6.20 (d, 1H, J=9.4 Hz, coum. CH), 5.58-4.90 (m, 2H+1H*2+2H, coum. benz. CH₂+2CH double bonds+triazole benz. CH₂), 4.56 (m, 2H, 2 glycerol backbone CH), 4.22-3.70 m, 2H*2+1H*2, NCH₂CH₂OP+2CH₂OP), 3.01 (s, 1H, NH), 2.32-2.07 (m, 4H, CH₂CO₂), 1.98 (m, 2H*2, CH₂CH═CHCH₂), 1.64 (m, 2H*2, CH₂CH₂CO₂), 1.25 (m, 48H, fatty chain CH₂), 0.87 (m, 6H, CH₃*2)

¹³C NMR (75.49 MHz, CDCl₃), δ (ppm): 172.8 (ester CO), 172.3 (ester CO), 161.4 (C_{q}O ether), 161.2 (coum. ester CO), 155.7 (coum. ester C_{q} alpha 0), 143.4 (CHₐᵣ), 142.5 (triazole C_{q}), 130.1 (fatty chain double bond CH), 129.6 (fatty chain double bond CH), 129.4-128 (trityl CHₐᵣ+coum. CHₐᵣ+triazole CHₐ), 113.4 (CHₐᵣ), 113.0 (Cqₐᵣ), 112.8 (CHₐᵣ), 102.1 (CHₐᵣ), 74.6 (trityl C_{q}), 70.4 (glycerol backbone CH next to the phosphorus), 69.5 (glycerol backbone CH remote from the phosphorus), 63.7 (CH₂ alpha OP, J=5.2 Hz), 62.1 (coum. benz. CH₂), 58.8 (ethanolamine CH₂O), 50.0 (triazole benz. CH₂), 49.2 (ethanolamine CH₂N), 34.0/34.1 (alpha ester CH₂), 32.0-22.8 (fatty chain CH₂), 14.2 (CH₃).

³¹P NMR (121.442 MHz, CDCl₃), δ (ppm): 2.01 ppm.

HRMS (ESI−) m/z calcd. Found: 1241.7233.

The intermediate compound as prepared in the preceding step (10 mg, 8.05 μmol, 1 equivalent) in 0.2 ml of anhydrous dichloromethane, and triethylsilane (3.74 mg, 32.2 μmol, 4 equivalents) in 0.1 ml of dry dichloromethane were placed in a 1 ml bottle under an argon atmosphere. Trifluoroacetic acid (3.67 mg, 32.2 μmol, 4 equivalents) in 0.1 ml of dry dichloromethane was then added to the mixture. The reaction turned yellow on addition of the acid, and was stirred for 3 h at room temperature. The reaction was monitored by UPLC (“Phenomenex Kinetex” 2.6μ PFP 100 Å 150×4.60 mm) with a UV detector (at 254 nm) to monitor the appearance of the free trityl group at 8.72 min) and a fluorescence detector (at 320 nm) to monitor the transition from the intermediate compound at 12.4 min to the product (XIVa-5) at 12.2 min.

Molar mass=999.6193 g·mol⁻¹.

HRMS (ESI−) m/z calcd. Found: 999.6179.

1. A synthetic glycerophospholipid corresponding to formula (I) below: 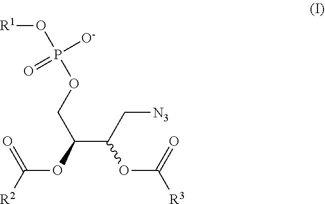
in which:
the group OR¹ is chosen from a hydroxyl group, a choline residue, an ethanolamine residue, a glycerol residue, a serine residue, an inositol residue, an inositol monophosphate residue, an inositol bisphosphate residue, an inositol trisphosphate residue, a glycerophosphate residue, a glucose residue, an inositolglycan residue, and a residue of any hydroxylated compound that is capable of forming a phosphodiester group in the sn-3 position belonging to a subclass of natural GPLs,
R² represents a mono- or polyunsaturated unsaturated aliphatic chain comprising at least 14 carbon atoms, and
R³ represents a saturated aliphatic chain comprising at least 10 carbon atoms.

2. The glycerophospholipid as claimed in claim 1, wherein R³ represents a saturated aliphatic chain comprising from 14 to 30 carbon atoms.

3. The glycerophospholipid as claimed in claim 1 wherein R² represents a mono- or polyunsaturated unsaturated aliphatic chain comprising from 16 to 24 carbon atoms.

4. The glycerophospholipid as claimed in claim 1 wherein the unsaturated aliphatic chain comprises from 1 to 6 unsaturations.

5. The glycerophospholipid as claimed in claim 1 wherein the glycophospholipid chosen from the compounds of formulae (Ia) to (Ie) shown in the following table:

| Name | Structural formula |
|---|---|
| Compound Ia | 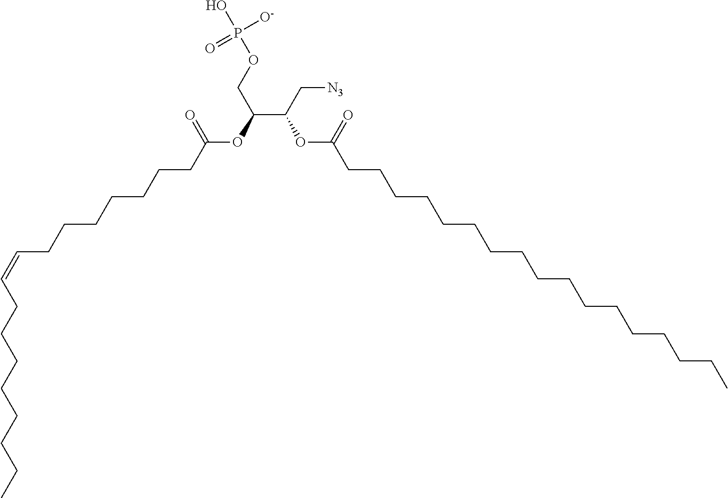 [SMILES: CCCCCCCC/C=C\CCCCCCCC(=O)O[C@@H](COP(=O)([O-])O)[C@H](CN=[N+]=[N-])OC(=O)CCCCCCCCCCCCCCCCC] |
| Compound Ib | 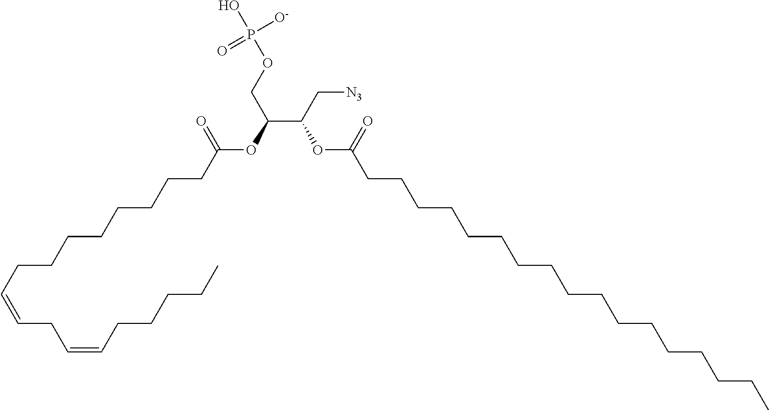 [SMILES: CCCCC/C=C\C/C=C\CCCCCCCCC(=O)O[C@@H](COP(=O)([O-])O)[C@H](CN=[N+]=[N-])OC(=O)CCCCCCCCCCCCCCCCC] |
| Compound Ic | 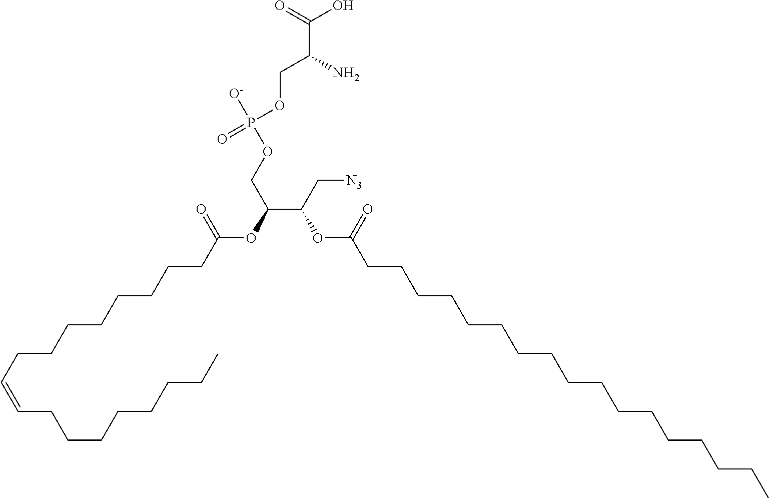 [SMILES: CCCCCCCC/C=C\CCCCCCCCC(=O)O[C@@H](COP(=O)([O-])OC[C@@H](N)C(=O)O)[C@H](CN=[N+]=[N-])OC(=O)CCCCCCCCCCCCCCCCC] |
| Compound Id | 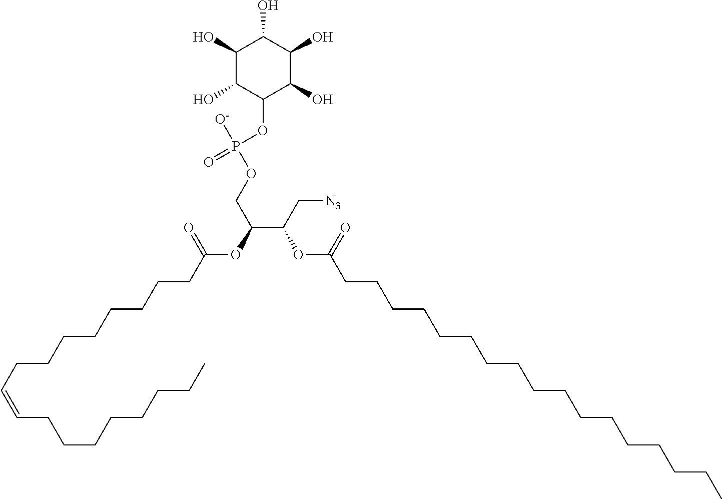 [SMILES: CCCCCCCC/C=C\CCCCCCCCC(=O)O[C@@H](COP(=O)([O-])OC1[C@H](O)[C@H](O)[C@@H](O)[C@H](O)[C@H]1O)[C@H](CN=[N+]=[N-])OC(=O)CCCCCCCCCCCCCCCCC] |
| Compound Ie | 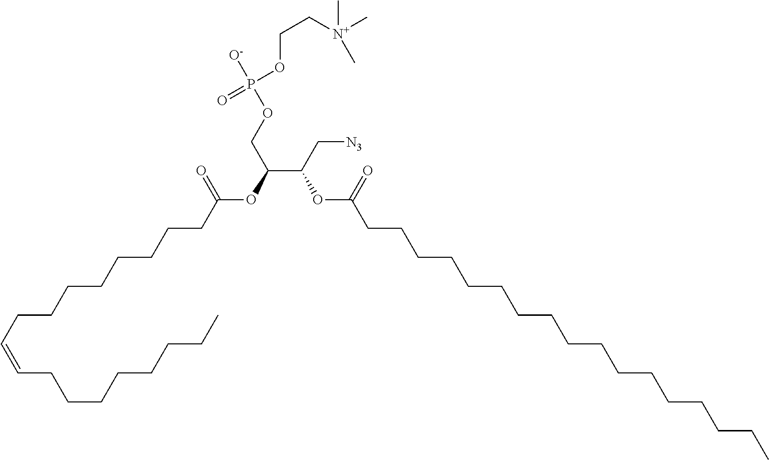 [SMILES: CCCCCCCC/C=C\CCCCCCCCC(=O)O[C@@H](COP(=O)([O-])OCC[N+](C)(C)C)[C@H](CN=[N+]=[N-])OC(=O)CCCCCCCCCCCCCCCCC] |

6. A process for preparing a glycerophospholipid of formula (I) as defined in claim 1, comprising:
i) a step of preparing a phosphotriester of formula (IV) comprising a diacetal function by functionalizing a diacetal derivative of (2S,3S)-butane-1,2,3,4-tetrol of formula (III) with a dialkyl halophosphate of formula (IV) in a basic medium, according to the following scheme: 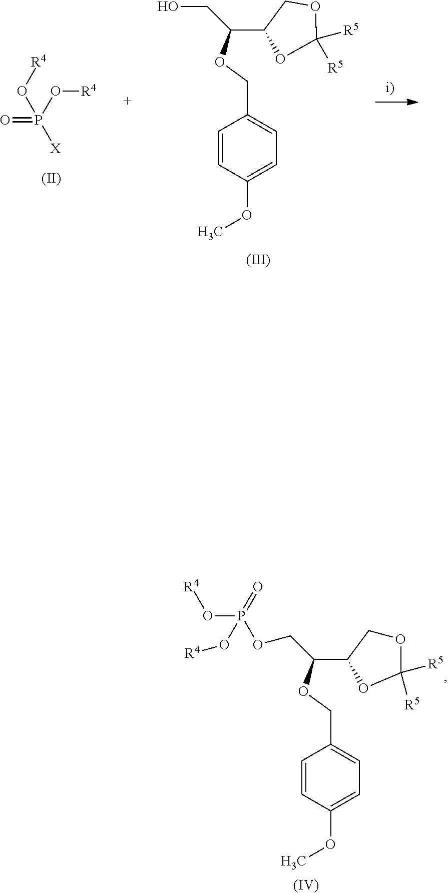
in which R⁴ represents a C₁-C₅ alkyl group or a benzyl group, and R⁵ represents a C₁-C₅alkyl group,
ii) a step of deprotection in acidic medium of the diacetal function of the phosphotriester of formula (IV) to form a diol of formula (V) comprising a primary alcohol, according to the following scheme: 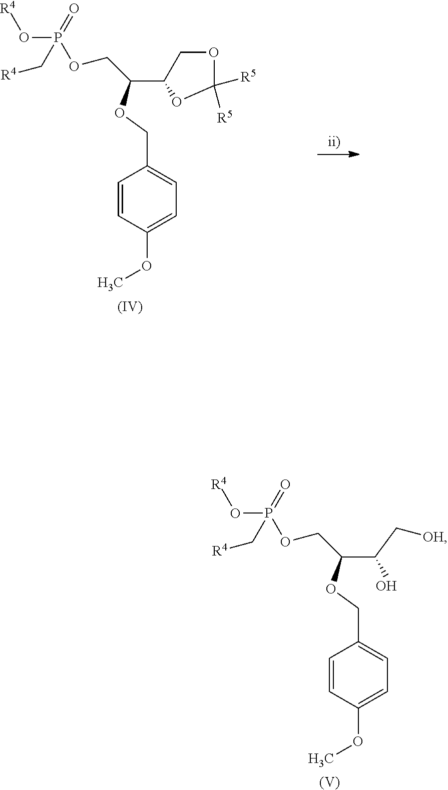
iii) a step of selective functionalization of the primary alcohol of the diol of formula (V) to form a monoalcohol of formula (VI) comprising a primary alcohol modified with a group R⁶, according to the following scheme: 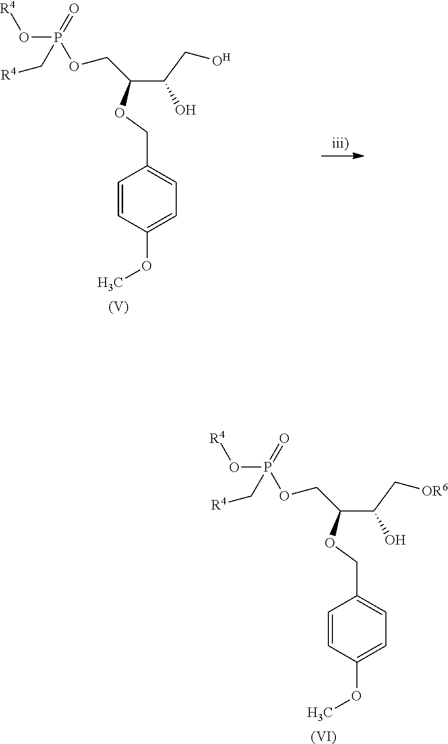
in which R⁶ is chosen from the following groups: tosyl, mesyl and triflate,
iv) a step of nucleophilic substitution of the function OR⁶ of the monoalcohol of formula (VI) to form an azide of formula (VII) comprising an alcohol function, according to the following scheme: 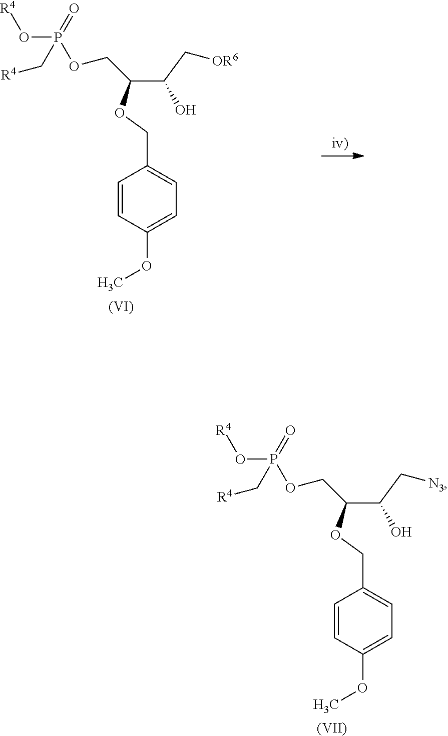
v) a step of esterification of the alcohol function of the azide of formula (VII) with a saturated fatty acid of formula (VIII) to form an azide of formula (IX) comprising a saturated fatty acid chain and an alcohol function protected with a para-methoxybenzyl group, according to the following scheme: 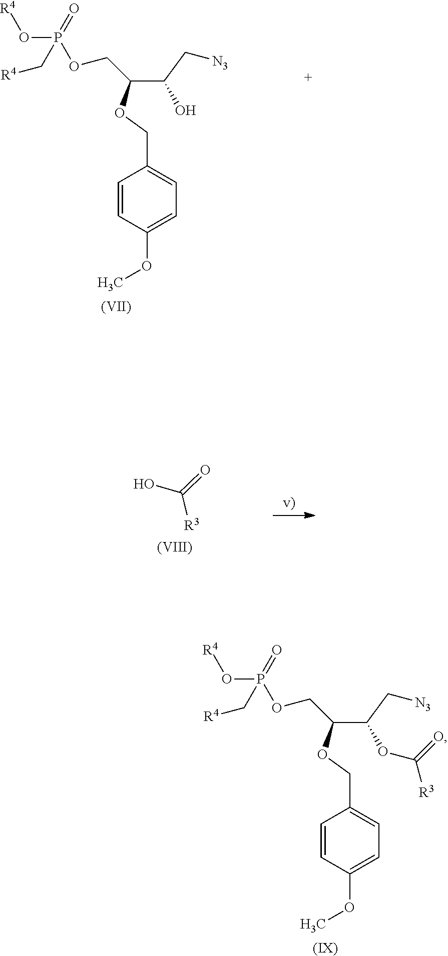
in which R³ is as defined in claim 1,
vi) a step comprising a sub-step vi-1) of deprotection of the para-methoxybenzyl group of the azide of formula (IX) to form an azide of formula (X) comprising an alcohol function, followed by a sub-step vi-2) of esterification of the alcohol function of the azide of formula (X) with an unsaturated fatty acid of formula (XI), to form an azide of formula (XII) comprising a saturated fatty acid chain, an unsaturated fatty acid chain, and a phosphotriester function, according to the following scheme: 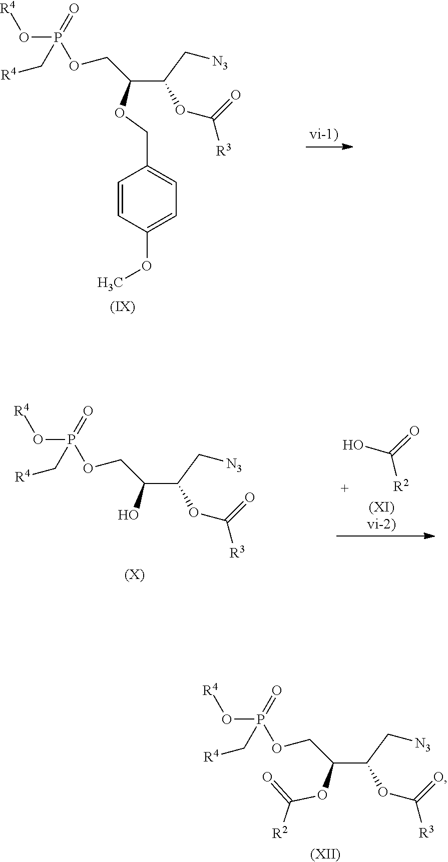
in which R² is as defined in claim 1, and
vii) a step of deprotecting the groups R⁴ to form a compound of formula (I) in which OR¹═OH, optionally followed by a step of preparing a compound of formula (I) in which OR¹≠OH.

7. The process as claimed in claim 6, characterized in that step iii) is performed in the presence of tosyl chloride, a base, and a tin-based catalyst.

8. The process as claimed in claim 6 wherein sub-step vi-1) is performed in the presence of 2,3-dichloro-5,6-dicyano-1,4-benzoquinone, and at a temperature ranging from −20° C. to 50° C.

9. A modified glycerophospholipid corresponding to formula (XIV) below: 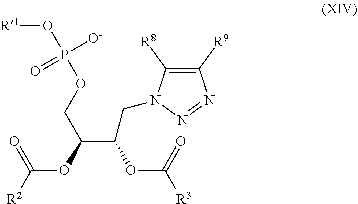
in which:
OR′¹ represents a group OR¹ as defined in claim 1, or a photoactivatable group,
R² and R³ are as defined in claim 1, and
R⁸ and R⁹, independently of each other, are chosen from a fluorophoric group, a photocrosslinkable group, a radiolabeling group and a hydrogen atom; or the groups R⁸ and R⁹ together form a group chosen from a fluorophoric group, a photocrosslinkable group and a radiolabeling group.

10. A process for preparing a modified glycerophospholipid of formula (XIV) as defined in claim 9, characterized in that it comprises at least one step A) of placing in contact a synthetic glycerophospholipid of formula (I) with an alkyne corresponding to formula (XV) below: 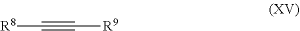
in which R⁸ and R⁹ are each independently of each other are, or together form, a group chosen from a fluorophoric group, a photocrosslinkable group and a radiolabeling group.

11. The process as claimed in claim 10, wherein step A) is performed after placing the synthetic glycerophospholipid (I) in contact with a biological medium and the process further comprises, prior to step A), a step a) of placing the synthetic glycerophospholipid (I) in contact with a biological medium, in particular in vitro or ex vivo.

12. (canceled)

13. (canceled)

14. (canceled)

15. A method of using the glycerophospholipid of formula (1) as defined in claim 1 in a medical application.

16. A method of using the modified glycerophospholipid of formula (X1V) as defined in claim 9 in a medical application.

17. A method of using the glycerophospholipid of formula (1) as defined in claim 1 as an in vitro or ex vivo diagnostic tool.

18. A method of using the modified glycerophospholipid of formula (X1V) as defined in claim 9 as an in vitro or ex vivo diagnostic tool.

19. A method of using the glycerophospholipid of formula (1) as defined in claim 1 as a medical tool or biological probe.

20. A method of using the modified glycerophospholipid of formula (X1V) as defined in claim 9 as a medical tool or biological probe.
